(19) United States
(12) Patent Application Publication
(10) Pub. No.: US 2007/0255355 A1
(43) Pub. Date: Nov. 1, 2007

(54) APPARATUS AND METHOD FOR SKIN TREATMENT WITH COMPRESSION AND DECOMPRESSION

(75) Inventors: Gregory B. Altshuler, Lincoln, MA (US); Andrei V. Erofeev, North Andover, MA (US); Robert R. Lopez, Boxford, MA (US); Liam O'Shea, East Greenwich, RI (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: PALOMAR MEDICAL TECHNOLOGIES, INC., Burlington, MA (US)

(21) Appl. No.: 11/697,667

(22) Filed: Apr. 6, 2007

Related U.S. Application Data
(60) Provisional application No. 60/744,400, filed on Apr. 6, 2006.

(51) Int. Cl. A61N 5/06

(52) U.S. Cl. 607/86

(57) ABSTRACT

The present invention generally provides methods and devices that allow more efficient delivery of a stimulus, such as optical radiation, to the skin. In many embodiments, negative and/or positive pressure is applied to one or more skin regions in order to maintain a skin target under tension so as to redistribute blood volume between the skin target and other skin segments. In many cases, such tension can cause a depletion of the volumetric blood content in the skin target (that is, in the blood vessels beneath a surface of the skin target), thereby facilitating delivery of radiation to the skin target.

### RELATED APPLICATIONS

The present application claims priority to U.S. provisional application entitled “Apparatus and Method for Skin Treatment with Compression and Decompression” having Ser. No. 60/744,400 filed on Apr. 6, 2006, and herein incorporated by reference.

### BACKGROUND OF THE INVENTION

The present invention relates generally to dermatological optical systems and devices, and more particularly, to such systems and devices with Electromagnetic Radiation (“EMR”) applicators capable of changing various skin parameters, such as blood distribution at the skin region.

Sources of electromagnetic radiation, particularly in the optical wavebands, are being increasingly utilized for various phototreatments of tissue (e.g., phototherapeutic and photocosmetic treatments). Some examples of phototreatment include light-based hair removal, treatment of various skin lesions (including pigmented and vascular lesions as well as acne), tattoo removal, facial skin improvement, fat and cellulite treatment, scar removal, and skin rejuvenation (including wrinkle reduction and improvement of tone and texture), odor redaction, and acne treatment. In performing such treatments, it is desirable that results are achieved efficiently and that risk of injury to the patient be minimized. However, some of the existing devices and methods do not treat tissue as efficiently and/or effectively as possible due to physical limitations such as, for example, scattering of EMR or insufficient contact with tissue.

Skin is a scattering medium, but such scattering is far more pronounced at some wavelengths than at others. Wavelengths preferentially absorbed by chromophores, such as melanin, that are frequently targeted in photocosmetic methods are also wavelengths at which substantial scattering occurs. This is also true for the wavelengths typically utilized for treating vascular lesions. Many wavelengths that are typically utilized for treatment are highly scattered and/or highly absorbed, which may limit the ability to selectively target body components, and in particular, may limit the depths at which treatments can be effectively and efficiently performed.

Further, current optical dermatology treatments can be inefficient since a large portion of the energy applied to a target region may be either scattered and may not reach the body component undergoing treatment, or may be absorbed in overlying or surrounding tissue to cause undesired and potentially dangerous heating of such tissue. If the efficiency of transmission of EMR in such treatments is low, larger and more powerful EMR sources may be required in order to achieve a desired therapeutic result and that additional cost and energy must be utilized to mitigate the effects of this undesired heating by surface cooling or other suitable techniques. Heat management for a more powerful EMR source may be a problem, because generally it requires expensive and bulky heat management mechanisms.

In some devices and treatments, the skin is compressed prior to treatment. This may help, for example, provide reliable contact with a treatment area, or may help remove blood from a volume of tissue to be treated by squeezing superficial blood vessels, which thereby enhances skin transparency for certain treatments and wavelengths. While pressing the device against the skin is effective in a bony area, such as arms, legs, shoulders, it can be problematic when treating areas with loose or voluminous skin, such as the abdomen, thighs and buttocks areas.

Therefore, a need exists for improved methods and apparatus for photocosmetic treatments, and in particular for optical dermatology treatments, which provide for improved contact with the target area and reduced light scattering, and can efficiently deliver radiation to a desired target volume at a selected depth.

### SUMMARY OF THE INVENTION

In one aspect, the invention provides a method for treating a volume of tissue which comprises applying a negative pressure to at least a portion of the volume of tissue (first portion), mechanically restraining a second portion of the volume of tissue, and irradiating the second portion of the volume with energy. By way of example, the second portion can be mechanically restrained by the surface of an energy-transmissive element through which the second portion is irradiated with the energy. In some cases, the energy-transmissive element, or optical element, can be a radiation-transmissive block. The radiant energy can be any suitable energy, such as electromagnetic radiation, acoustic energy, electric current, and heat. The first and second portions do not overlap in some embodiments. In other embodiments, these volume portions at least partially overlap. The method can further include cooling the volume of tissue. For example, the cooling step can comprise cooling the end of the optical element that is in contact with the surface of the volume of tissue.

In some embodiments, the energy can be electromagnetic radiation and the method further comprises selecting one or more wavelengths of the radiation so as to perform any of acne treatment, skin rejuvenation, hair removal, cellulite treatment, fat reduction, wrinkle and scar reduction, collagen regeneration, tattoo removal, and treatment of pigmented and vascular lesions. The electromagnetic radiation can include at least one wavelength in a range of about 300 nm to about 11,000 nm, or preferably at least one wavelength in a range of about 300 nm to about 3,000 nm. Electromagnetic radiation can be delivered to the volume of tissue at a power density in a range of about 1 mW/cm² to about 1000 W/cm², or preferably in a range of about 100 mW/cm² to about 10 W/cm² to the volume of tissue. The electromagnetic radiation can be delivered to the volume of tissue at a fluence in a range of about 1 J/cm² to about 1000 J/cm², or preferably in a range of about 10 J/cm² to about 500 J/cm².

In a related aspect, the application of negative pressure can stretch the volume of tissue. For example, the volume of tissue can be stretched for an amount of time sufficient to reduce the amount of blood in that volume of tissue. For example, negative pressure can be applied for a duration of about 1 milliseconds to about 2 seconds. Negative pressure can be applied in a range of about 6.7×10³ Pa to about 1×10⁵ Pa, or preferably in a range of about 23×10³ Pa to about 41×10³ Pa. The negative pressure can be released after application of the radiation to the volume of tissue. The method can also include the step of monitoring the negative pressure to ensure it remains within a desired range, e.g., below a pre-determined threshold. The step of monitoring the negative pressure can comprise adjusting the pressure based on a selected treatment for the volume of tissue.

The negative pressure can be applied such that the volume of tissue is stretched in a first direction. The negative pressure can be applied such that the volume of tissue is stretched in a second direction. In some cases, the negative pressure can be alternatively to the volume of tissue applied along two different directions so as to alternatively stretch the volume of tissue along the first direction and then the second direction.

In some embodiments, the method further includes applying a positive pressure to at least a third portion of the volume of tissue. The third portion can overlap at least one of the first and second portions. Alternatively, the third portion does not overlap the first or second portions. In related aspects, pressure applied to a region of the skin can alternate between positive and negative pressure.

In some embodiments, the energy-transmissive element can be moved from one volume to another volume of tissue by sliding the element over the skin surface. During the transition between the two volumes, the intervening tissue can be irradiated with energy. Alternatively, the step of moving can be accomplished by stamping each volume of tissue being treated.

In some embodiments, the method includes compressing a third portion of the volume of tissue. At least some of the third portion of the volume of tissue can be contiguous with at least some of the second portion of the volume of tissue that is mechanically restrained. The method can further include mechanically restraining a third portion of the volume of tissue, and irradiating the third portion of the volume of tissue with electromagnetic radiation, wherein the second and third portions of the volume of tissue are not contiguous.

In some embodiments, the step of irradiating can further include simultaneously irradiating a plurality of portions of the volume of tissue, wherein each portion is spaced at a distance from the other portions.

In another aspect, a photocosmetic method is disclosed that comprises placing an optically transmissive surface in proximity of a skin region, applying a negative pressure to the skin region in order to draw a portion thereof into contact with the optical surface so as to redistribute blood volume between the skin portion in contact with the optical surface and the remainder of the skin region, and applying radiation through the surface to the skin portion. The redistribution of the blood volume can be characterized by a decrease in volumetric blood concentration at the skin portion in contact with the surface and a respective increase in volumetric blood concentration in the remainder of the skin region. The method can further comprise selecting the negative pressure such that the skin portion in contact with the surface substantially covers the surface, and/or substantially conforms to a topographical profile of the surface. The method can further comprise cooling the surface. In some cases, the negative pressure can cause stretching of the skin portion that is in contact with the surface.

In another aspect, the invention discloses a method of dermatological treatment, which comprises applying negative pressure to a plurality of surface skin segments within a skin region so as to redistribute blood within the region, applying radiation to the skin region, wherein the blood redistribution causes a non-uniform absorption of the radiation across the skin region. The non-uniform absorption can be an increased absorption at one or more skin targets located below the surface of the skin segments. The method can further comprise monitoring the negative pressure applied to the skin segments. The negative pressure and radiation can be adjusted based on a desired radiation pattern corresponding to a desired treatment of the skin region.

In yet another aspect, a dermatological treatment method is disclosed which comprises placing an optical surface in proximity of a skin region containing a skin target, the optical surface being at least partially surrounded by a negative pressure chamber, applying a negative pressure to the skin region so as to draw the skin target into contact with the optical surface causing redistribution of blood volume between the skin target and the remainder of the skin region, and applying radiation through the surface to the skin target.

In another aspect, a method for treating a volume of tissue is provided comprising applying a negative pressure to at least a first portion of the volume of tissue, compressing a second portion of the volume of tissue, and irradiating the second portion of the volume with electromagnetic radiation.

In another aspect, the invention provides a dermatological device which comprises an optical element adapted for contact at one end thereof with a skin target, and a negative pressure chamber at least partially surrounding the end of the optical element, wherein the negative pressure chamber is adapted to apply a negative pressure to one or more locations of a skin region so as to draw the skin target into compressive contact with the end of the optical element and to cause a depletion of blood volume within the skin target. The negative pressure applied to the skin region can be in a range of about 6.7×10³ Pa to about 1×10⁵ kPa. The negative pressure chamber can be adapted to apply a negative pressure along an axial direction to the skin. In some embodiments, the axial negative pressure can cause a transverse stretching of the skin target. The negative pressure chamber can include a plunger for generating a negative pressure therein, or the negative pressure chamber can be coupled to a source of negative pressure.

In some embodiments, the device further includes means for controlling the negative pressure. The device can further include a pressure sensor and a feedback loop between the pressure sensor and the source of negative pressure. The device can further include a radiation source capable of irradiating through the optical element. The feedback loop can be adapted to activate the radiation source in response to a detected pressure. Further, the feedback loop can activate a pressure safety value if the detected pressure in not within a desired range. The device can also include a pressure release valve and/or a pressure controller.

In another aspect, a dermatological device is disclosed, which comprises a radiation-transmissive element configured to be in contact with a skin target for applying electromagnetic radiation thereto, a channel extending from a proximal end adapted for coupling to a pressure source to a distal end opening to a pressure chamber configured to apply a pressure (a positive or a negative pressure) to a skin region containing at least one skin portion offset from the skin target. At least a portion of the element is located within the pressure chamber. The distal end of the channel can be axially offset from the distal end of the element, or the distal end of the channel is substantially flush with the distal end of the element. In some embodiments, the distal end of the channel can be surrounded by an inflatable cuff capable of increasing tension of the skin target.

In another aspect, the invention discloses an adapter for use with a photocosmetic device, comprising a pressure applicator assembly adapted to removeably and replaceably couple to a distal end of a waveguide of the device, where the coupling comprises a seal between the assembly and the distal end. The assembly can comprise a negative pressure chamber at a distal end thereof adapted for coupling to a skin portion to apply a negative pressure thereto, and at least one channel extending from a proximal end adapted for coupling a source of negative pressure to the distal end having an opening to the chamber. The pressure applicator can further include a pressure sensor, which can be coupled to a pressure control valve.

In another aspect, a method of treating the skin is disclosed, which comprises placing a surface of a radiation-transmissive element in contact with a skin target, applying a negative pressure to a periphery of the skin target so as to cause stretching thereof, and applying radiation through the surface to the skin in contact therewith.

In yet another aspect, the invention discloses a dermatological device, which comprises an optical waveguide adapted for contact at one end thereof with a skin target, a skin pressure applicator coupled to the end of the waveguide, where the applicator comprises a first channel adapted for coupling at a proximal end to a source of positive pressure and for applying at a distal end a positive pressure to a first skin region, a second channel adapted for coupling at a proximal end to a source of negative pressure and for applying at a distal end a negative pressure to a second skin region. The skin regions can be offset relative to the skin target. For example, the skin regions can partially surround the skin target. The pressures can be selected to hold the skin target under tension in contact with the end of the optical waveguide.

In another aspect, a dermatological device is disclosed, which comprises a housing providing an optical path extending from a proximal end thereof to a distal end for applying radiation to a skin region and a skin pressure applicator coupled to the distal end of the housing for applying pressure to the skin region. The pressure applicator can comprise a pressure mask (e.g. a mask formed of a radiation-transmissive material) having a plurality of openings to allow application of pressure to a plurality of locations of the skin region so as to non-uniformly redistribute blood volume within that region. The pressure applicator can further comprise a pressure chamber to which the mask is coupled. Negative or positive pressure can be applied via the pressure chamber to the skin located below the mask openings.

### BRIEF DESCRIPTION OF THE DRAWINGS

FIG. 1A schematically depicts a pressure chamber with an optical element positioned therein where the tip of the optical element is flush with the open end of the pressure chamber,

FIG. 1B schematically depicts utilizing the pressure chamber of FIG. 1A to apply negative pressure to the skin,

FIG. 2A schematically depicts a pressure chamber with an optical element positioned therein where the tip of the optical element is recessed within the open end of the pressure chamber,

FIG. 2B schematically depicts utilizing the pressure chamber of FIG. 2A to apply negative pressure to the skin,

FIG. 3A schematically depicts a pressure applicator attached to an optical mask with a plurality of holes,

FIG. 3B schematically depicts utilizing the pressure applicator of FIG. 3A to apply negative pressure to a plurality of skin segments,

FIG. 4 schematically depicts a dermatological optical system in accordance with one embodiment of the invention,

FIG. 5A is a schematic cross-sectional view of a pressure applicator in accordance with one embodiment of the invention that is coupled to a radiation waveguide of a dermatological handpiece,

FIG. 5B schematically depicts utilizing the pressure applicator of FIG. 5A to apply a negative pressure to a skin region,

FIG. 5C schematically depicts that the pressure applicator of FIG. 5A can be utilized to maintain a skin target under tension in contact with a distal end of the waveguide,

FIG. 6 is a schematic cross-sectional view of a pressure applicator according to another embodiment of the invention coupled to a radiation waveguide at a distal end of a dermatological handpiece,

FIG. 7 is a schematic cross-sectional view of a pressure applicator according to another embodiment of the invention,

FIG. 8 is a schematic cross-sectional view of a pressure applicator according to another embodiment of the invention,

FIG. 9 schematically depicts an exemplary implementation of a pressure applicator according to one embodiment of the invention, which is designed to be coupled to a distal end of a dermatological handpiece,

FIG. 10 schematically depicts an exemplary implementation of a pressure applicator according to another embodiment of the invention,

FIG. 11A is a schematic cross-sectional view of a pressure applicator according to another embodiment of the invention having a pressure mask that generating a plurality of positive and negative pressure zones in a skin target,

FIG. 11B is schematic top view of the pressure mask of the applicator of FIG. 11A,

FIG. 12 schematically depicts the use of the pressure mask of FIG. 11A applying negative pressure to certain locations on a skin target and positive pressure to other locations thereof.

### DETAILED DESCRIPTION

The present invention generally provides methods and devices that allow more efficient delivery of a stimulus, such as electromagnetic radiation, to the skin. In many embodiments, negative and/or positive pressure can be applied to one or more skin regions in order to maintain a skin target under tension. In some embodiments, such tension can cause a depletion of the volumetric blood content in the skin target (that is, in the blood vessels beneath a surface of the skin target), thereby facilitating delivery of radiation to the skin target. In other embodiments, the tissue can be physically stretched in one or more directions. Still other embodiments allow the manipulation of other physical properties of the tissue and/or combinations of physical properties of the tissue. As discussed in more detail below, other advantages of maintaining the skin target under tension include bringing some tissue structures of interest closer to the skin surface, and hence closer to a radiation source, and minimizing heat flux due to blood perfusion.

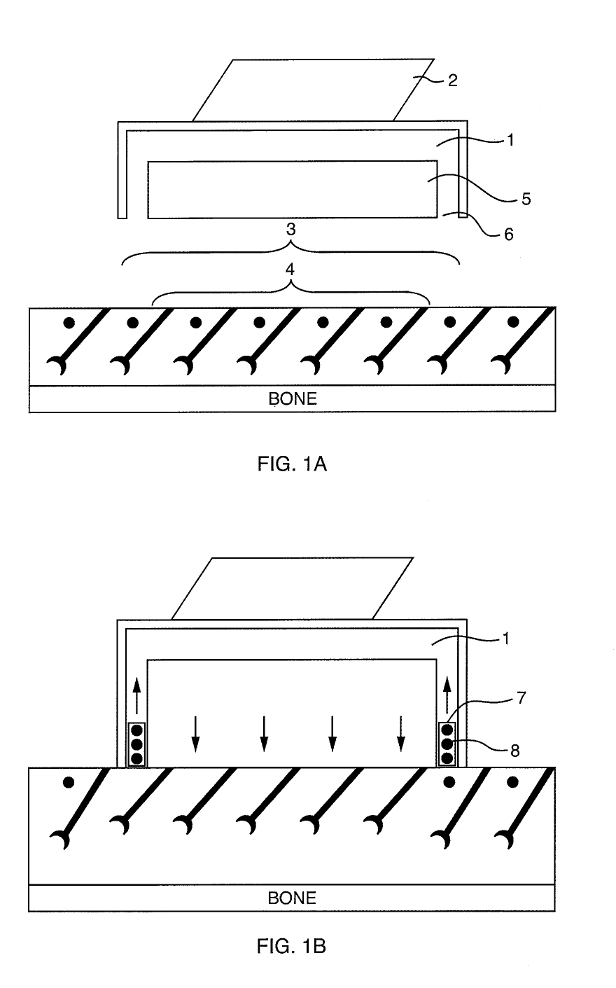

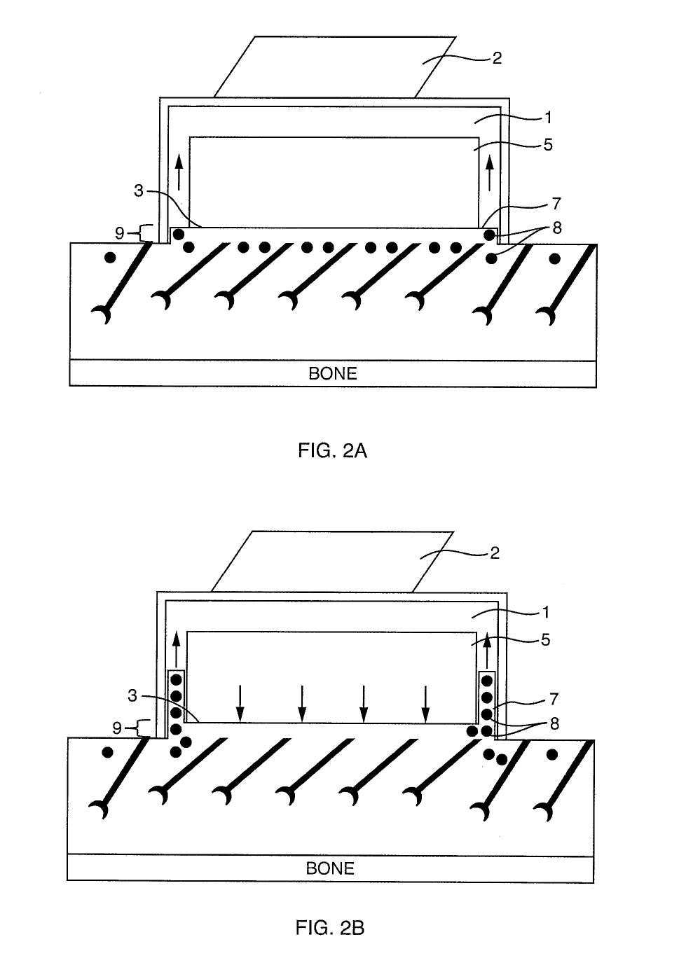

With reference to FIGS. 1A-B and 2A-B, in one exemplary embodiment of a dermatological device and method according to the teachings of the invention, a negative pressure generated in a chamber 1 of a handpiece 2 is applied to a skin portion 3 containing a skin target 4 so as to lift the skin target 4 towards a distal tip of the handpiece's EMR emitting element 5, a sapphire waveguide in this embodiment. The tip 6 of the EMR emitting element 5 can be flush with the edge of the pressure chamber as shown in FIGS. 1A&B. Alternatively, the tip 6 can be recessed into the chamber as shown in FIGS. 2A&B, and described further below, such that, during operation, the tissue 3 is drawn into a recess portion 9 of chamber 1. During the lifting of the skin, uncompensated internal blood pressure inside the skin opens up blood vessels, thereby causing a several fold increase in the blood volumetric content of the skin. Positioning the tip 6 of the EMR emitting element 5 inside the chamber 1 enhances the lifting of the skin. Therefore, position of the tip 6 of the EMR emitting element 5 can be adjusted depending on the desired application or patient characteristics, such as skin type. Once the skin target is in contact with the light guide's distal tip, the tip applies a positive pressure to the skin target, which redistributes blood 8 from the skin vessels beneath the distal tip (i.e., the vessels beneath the surface of the skin target) to unconstrained portion of the skin (i.e., the skin portion 3 that is drawn toward the chamber 1 and in many cases into the side portion 7 of chamber 1, which extends about the perimeter of EMR emitting element 5 in the present embodiment). (Inless otherwise specified, positive and negative pressures as used herein refer to relative pressures.)

In this embodiment, the tip or surface of the waveguide (or other EMR-transmissive elements in other embodiments) may act as a mechanical restraint that constrains the volume of tissue. When the volume of tissue is in physical contact with the tip or surface of the waveguide, it can then be further manipulated, e.g., by stretching the tissue, compressing the tissue, or altering the magnitude and/or direction of the pressure.

For example, as the unconstrained skin portion continues to be drawn into the negative pressure chamber, it causes additional deformation (e.g., stretching) of the skin target (in this case, the constrained portion of the skin) that is already in contact with the light guide's distal tip. In this manner, the skin target is maintained under tension while concurrently its blood content is lowered. Then, radiation, e.g., cosmetic and/or therapeutic radiation, can be applied via the distal tip of EMR emitting element 5 to the skin target.

The lower blood content will enhance the skin transparency for certain light wavelengths (e.g., blue light bands (380-450 nm) and green light bands (500-610 nm)), and hence can improve the cosmetic and/or therapeutic effects of the applied EMR in methods and devices that employ such wavelengths. In addition, the compression and stretching of the skin decreases light scattering and reduces the thickness of the basal membrane, which helps light penetrate deeper into the tissue by reducing the optical density of the basal membrane and bringing tissue structures located at depth closer to the light source. Stretching of the skin in the treatment area also allows more efficient cooling by reducing the conduction path from the chilled waveguide to the heat sensitive elements of the skin. Keeping the skin in tension in the treatment area restricts blood flow thereby minimizing heat flux due to blood perfusion, which allows increased energy delivery while maintaining patient comfort as well as safety. Note that, in embodiments where negative pressure is applied around the perimeter of the EMR-emitting element and the EMR-emitting element is also compressed against the tissue, blood will be forced from volume of tissue being treated, while negative pressure alone (as in the case where a volume of tissue is drawn into a chamber during treatment and is not compressed significantly) will cause an increase in the amount of blood in the volume of tissue being treated.

Further, in many embodiments, the configuration of the pressure chamber is selected so as to optimize the deformation of the skin target. By way of example and as discussed further below, in many embodiments the EMR emitting element is designed to emit radiation from the device to the tissue, and can be any structure suitable for that purpose(e.g., a sapphire, plastic, glass or micro-porous quartz waveguide, window or other suitable structure) The EMR-emitting element has a desired cross-sectional shape (e.g., circular, square or rectangular) with a major dimension in a range of, e.g., about 10 mm to about 50 mm across the area from which EMR is emitted. Further, the skin-contacting surface of the waveguide's tip can have any desired profile, such as concave, convex, flat, conical or trapezoidal. Additionally, the waveguide's surface-contacting tip can include features such as channels, serrations, arranged in various patterns. As discussed further below, in some embodiments, such features can form positive and/or negative pressure passages for generating a variety of skin stress islets. In many of such embodiments, the pressure chamber wraps partially or fully around the EMR emitting element with a spacing between an edge of the tip of the element to a part of the negative pressure chamber closest to the tip (e.g., a wall of the chamber) lying in a range of about, e.g., 0 to about 10 mm.

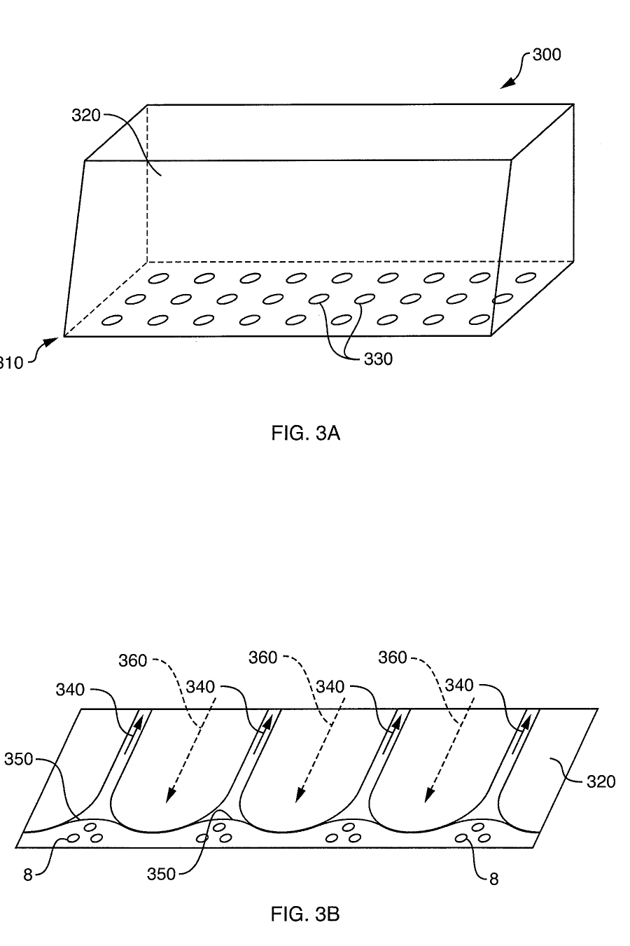

In another embodiment, the pressure is applied to the skin portion at a plurality of surface skin segments within a skin region so as to redistribute blood within the region. In one exemplary embodiment shown in FIG. 3A, a skin pressure applicator 300 can be coupled to the distal end of the housing for applying pressure to the skin region. The pressure applicator 300 can comprise a mask 310, which is capable of being permanently or removeably coupled to a pressure chamber 320. The mask can be made of an optically transmissive material or a partially optically transmissive material and can have plurality of openings 330 of various sizes and shapes to allow application of pressure to a plurality of locations of the skin region so as to non-uniformly redistribute blood volume or otherwise manipulate the tissue at regular intervals along the surface of the tissue. (Alternatively, the intervals may be irregular and many other patterns and shapes may be used.) The pressure chamber can be coupled to a negative pressure source allowing negative pressure 340 to be applied to the plurality of skin segments 350 lifting the skin and increasing blood volumetric content at these segments as shown in FIG. 3B. The application of the negative pressure to the plurality of skin segments 350 results in non-uniform redistribution of the volumetric blood content. Once the skin region is in contact with the surface of the mask, the mask applies a positive pressure to the skin region, which further redistributes blood and stretches the skin. When radiation 360 is applied to the skin region, this blood redistribution causes a non-uniform absorption of the radiation across the skin region. The non-uniform absorption can result in increased absorption at one or more skin targets located below the skin surface by decreasing blood volume at a plurality of regions between the skin segments and increasing skin transparency. The mask also enables fractional treatment of the treatment area, e.g., irradiating selected skin portions that are separated from one another by non-irradiated portions. Fractional treatment protects the patient's skin and facilitates healing of damage thereto, while still permitting the desired therapeutic effect to be achieved. In addition, the pressure mask can be used to redistribute blood so that different types of treatment can be accomplished during a single radiation exposure as described below.

In many embodiments, including the embodiments shown in FIGS. 1A-3B, the pressure chamber is coupled to a negative pressure source. The applied negative pressure can be in a range of about 2 inch Hg (6.7×10³ Pa) to about 30 inch Hg (1×10⁵ Pa), preferably in the range of about 5 inch Hg (16.9×10³ Pa) to about 20 inch Hg (67.7×10⁵ Pa), or more preferably in the range of about 7 inch Hg (23×10³ Pa) to about 12 inch Hg (41×10³ Pa). However, the optimum pressure level will vary depending on the type of treatment, skin type as well as pain tolerance of the patient. At some point, the benefits achieved by increasing the amount of negative pressure reaches a point of diminishing return. For example, in an exemplary embodiment, the negative pressure can be maintained at, above or below about 10 inch Hg. However, at pressures above 10 inches Hg, there is essentially no further displacement of tissue, while the displacement of tissue as a function of negative pressure is roughly linear from 0 to 10 inches Hg. Note also, that the relationship between tissue displacement and negative pressure is more predictable and results in less deviation when a lotion is first applied to the surface of the tissue as opposed to applying negative pressure to tissue to which no lotion as been applied.

Thus, although not required, it may be preferable to apply lotion to the tissue prior to treatment. In some embodiments, a topical substance can be placed on the skin target prior to treatment or application of pressure. Additionally, such a lotion could provide other benefits, such as improved efficiency of transmission of EMR, in the case of an index matching lotion. By way of example, the topical substance can enhance the coupling of the radiation to the skin and can also allow for a more uniform application of pressure to the skin target. By way of example, the topical substance can be selected from the group consisting of lotion, cream, wax, film, water, alcohol, oil, gel, powder, aerosol, and granular particles. The topical substance can achieve at least one of moisturizing skin, UV protection, tanning skin, improving skin texture, improving skin tone, reduction and/or prevention of cellulite, reduction and/or prevention of acne, wrinkle reduction and/or prevention of wrinkles, reduction of scars, reduction and/or prevention of vascular lesions, reduction in pore size, oil reduction in sebum secretion, skin elasticity improvement, reduction in sweat secretion, reduction and/or improvement of odor, body hair reduction or removal, and stimulation of hair growth.

A lotion can be applied to the treatment region to stabilize the effect of the negative pressure enhancing the thermal and/or optical contact with the optical element. The lotion dispensed on the skin can contain both skin beneficial ingredients and compounds designed to improve the thermal and optical contact between handpiece and skin. By depositing a lotion designed to improve thermal and optical contact prior to laser irradiation, improved safety and efficacy can be achieved. The lotion can be cooled to make the treatment more comfortable for the user. The lotion will provide lubrication, which allows the handpiece to be either easily scanned across the skin surface, or the negative pressure chamber more easily released from the skin reducing redness of the skin.

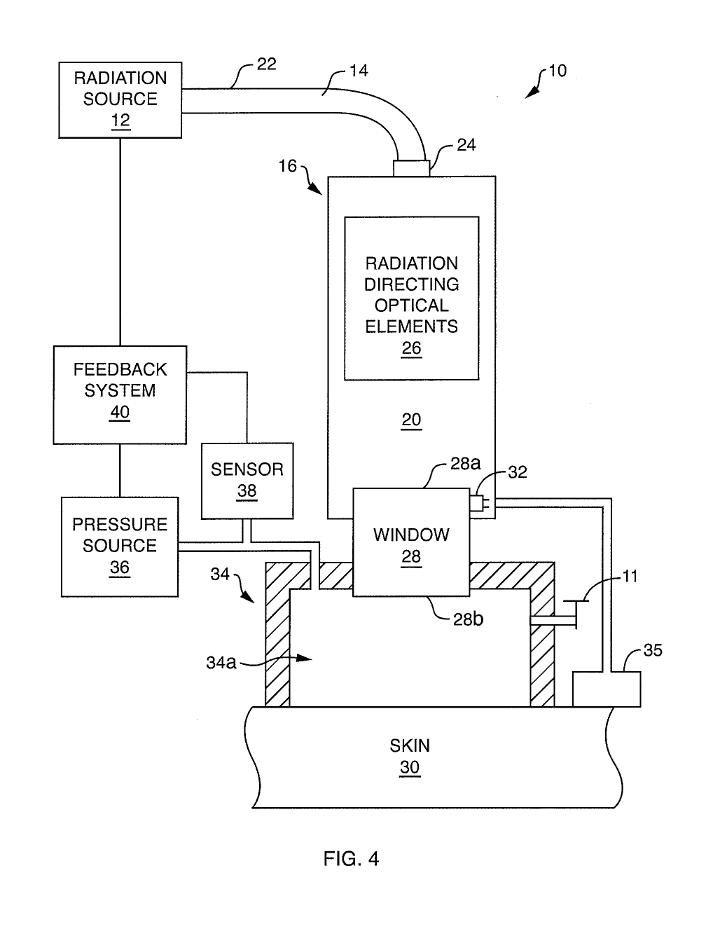

FIG. 4 schematically depicts a dermatological optical system 10 in accordance with one embodiment of the invention that includes a radiation source 12 for generating radiation, which is coupled via an optical fiber 14 to a handpiece 16 that can, in turn, apply the radiation to a subject's skin 18 in a manner discussed below. Radiation source 12 can be any suitable source providing radiation in a desired wavelength range, such as those listed further below. In other embodiments, the radiation source can be incorporated within the handpiece, e.g., in a manner described in U.S. patent application Ser. No. 10/154,756 filed May 23, 2002, which is incorporated herein by reference. In this embodiment, the handpiece includes a handheld housing 20 that can be coupled via the optical fiber 14, which is disposed in an umbilical cord 22, to the radiation source. The umbilical cord can contain other optical, thermal and/or electrical communication paths between the handheld device 16 and the radiation source 12.

The handheld device 16 can include a port 24 at a proximal end thereof for coupling to the optical fiber 14 so as to direct the radiation from the source to a plurality of optical elements 26, such as lenses, that can in turn direct the radiation to a radiation waveguide 28, e.g., in the form of a radiation-transmissive block. In this embodiment, the waveguide 28 comprises a sapphire block that extends from a proximal surface 28a to a distal surface 28b, which is adapted for contact with the skin. The radiation directed by the optical elements 26 to the proximal surface 28a of the waveguide passes through the waveguide to be applied via the waveguide's distal surface 28b to the skin 30. In this embodiment, the waveguide is thermally coupled via a side surface thereof with a cooling plate 32, which is cooled via the flow of a cooling fluid, e.g., water, through one or more inner passages thereof (not shown). In this manner, the waveguide's distal surface can be cooled, which in turn results in cooling of the skin surface that is in contact therewith.

With continued reference to FIG. 4, the exemplary handpiece 16 further includes a skin pressure applicator 34 coupled to its distal end that surrounds the radiation waveguide 27. As discussed further below, the pressure applicator 34 includes a pressurization cavity (chamber) 34a in fluid communication with a source of pressure 36 (e.g., positive or negative pressure) for applying negative and/or positive pressure to one or more skin regions so as to cause a desired deformation of a skin target in contact with the waveguide's distal surface. By way of example and as discussed below, in some embodiments, the pressure applicator can apply a negative pressure to skin segments on the periphery of a skin target to bring the skin target into contact with the distal surface 28b of the waveguide 28 and to maintain the skin target under tension while in contact with that surface. This can cause a redistribution of the volumetric blood content between the skin target and the surrounding skin, thereby facilitating application of radiation to the skin target. In some embodiments, the pressurization chamber is designed to partially or fully wrap the perimeter of the waveguide with the spacing from an edge of the waveguide's tip to the closest part of the pressurization chamber lying in a range of about 0 to about 10 mm.

With continued reference to FIG. 4, in this embodiment, a pressure sensor 38 can monitor the pressure in the chamber 34a and send the pressure reading to a feedback system 40, which ensures that the pressure remains within a predefined range. By way of example, if the pressure begins to deviate from a selected range, the feedback system can send a control signal to the pressure source to disable it, while in some cases concurrently activating a safety valve (not shown) to expose the chamber to atmospheric pressure. In some cases, the feedback system can also communicate with the radiation source, e.g., to activate the source once a desired pressure in the chamber is achieved. Further, a pressure relief valve 11 that can be utilized to bring the pressure within the chamber to atmospheric level once application of radiation to a skin target is accomplished. This allows readily disengaging the handpiece from one skin segment and moving it to another. In some embodiments, a lotion dispenser 35 can be coupled to the optical system 10 for dispensing lotion prior to treatment.

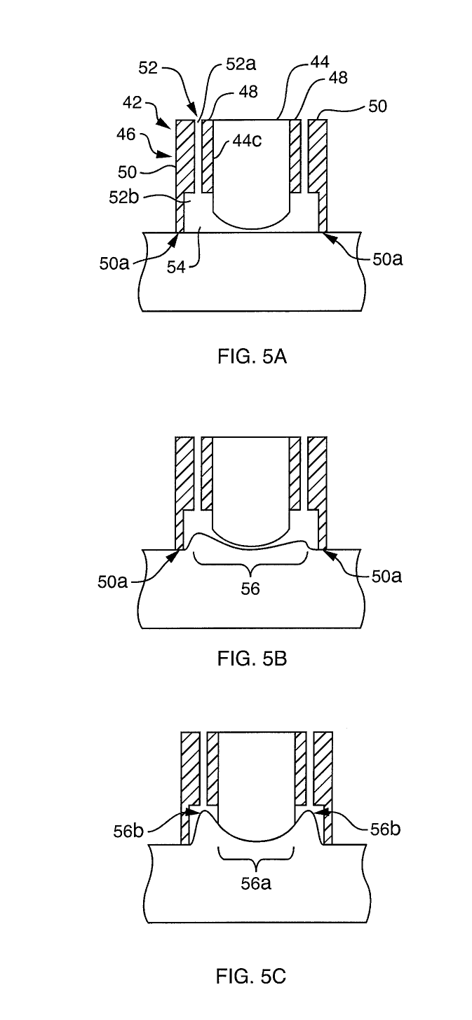

By way of example, FIG. 5A schematically depicts a cross-sectional view of one exemplary implementation of a pressure applicator 42, which is attached to a distal portion of the handpiece surrounding a radiation waveguide 44. The pressure applicator includes a housing 46 formed of an inner wall 48, which is disposed adjacent a side surface 44c of the waveguide, and an outer wall 50. A pressurization passage 52 (herein also referred to as a pressurization channel) is formed between the inner and outer walls. A proximal end of the passage 52 includes an opening 52a for coupling to a source of negative or positive pressure (not shown), and a distal end of the passage includes an opening 52b that allows fluid communication (air flow) between the passage and a cavity 54 via which a positive or negative pressure can be applied to the skin. The outer wall 50 includes a section that extends longitudinally from the proximal end of the passage 52 to its distal end, and a narrower section that extends longitudinally beyond the passage's distal end to provide a tip 50a adapted for contact with the skin. In this embodiment, the skin-contacting surface of the waveguide is recessed relative to the skin-contacting tip 50a of the pressure applicator to form the pressurization cavity 54.

In use, the tip 50a of the pressure applicator can be pressed against the skin to form a seal therewith, thereby turning pressurization cavity 54 into a pressurization chamber. A positive or negative pressure can then be applied via the passage 52 to the pressurization chamber, and consequently to the skin. By way of example, with reference to FIG. 5B, upon application of a negative pressure, a skin segment 56 encircled by the tip 50a is drawn up into the negative pressure chamber. As the negative pressure is maintained, the skin segment 56 continues to be lifted up toward the waveguide such that finally a portion thereof 56a (skin target) comes into contact with the tip of the waveguide. The unconstrained portions 56b of the skin segment surrounding the skin target (i.e., the portion whose movement is not constrained by contact with the waveguide) continues to be drawn up toward the opening 52b of the passage 52 to further apply a tensile force to the skin target. Once a steady state is achieved, the skin target (the skin beneath the waveguide and in contact therewith) is held in tension by a combination of a normal force applied by the waveguide and an opposing force exerted to the periphery of the skin target (unconstrained skin portion) by the applied negative pressure. In this manner, the volumetric blood content of the skin target can be decreased. In other words, a redistribution of the blood volume between the skin target and the unconstrained portion of the skin surrounding the target can be achieved corresponding to a decrease in the blood content of the skin target and an increase in the blood content of the unconstrained skin portion.

Referring again to FIG. 5A, in some embodiments, a width w of the inner wall is in a range of about 0 (that is, the sidewall of the waveguide forms a wall of the passage 52) to about 10 mm.

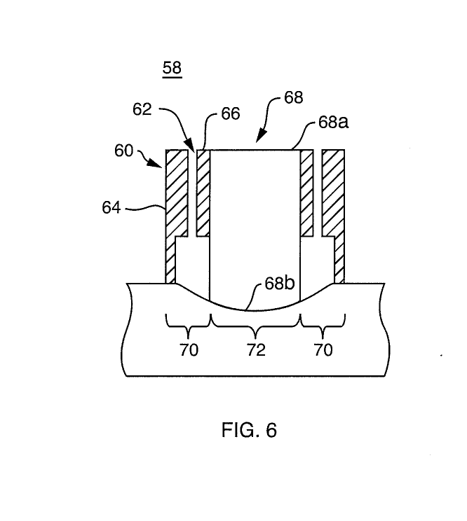

FIG. 6 schematically depicts a pressure applicator 58 according to another implementation that includes a housing 60 with a passage 62 formed between external wall 64 and internal wall 66, which surrounds a radiation waveguide 68 that extends from a proximal surface 68a to a distal surface 68b. In this embodiment, the distal surface 68b of the waveguide extends beyond a distal tip 64b of the external wall 64. Although the applicator 58 can be utilized to apply positive or negative pressure to the skin, in this illustration, it is employed to apply a positive pressure to a skin segment 70 surrounding a skin target 72 that is in contact with the waveguide's distal surface.

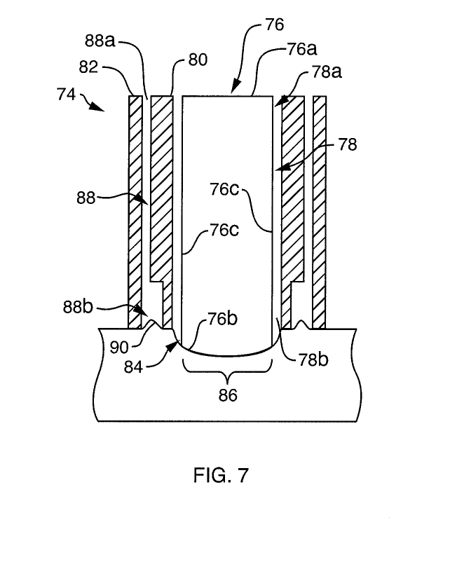

FIG. 7 schematically depicts a pressure applicator 74 in accordance with another embodiment that is coupled to a light guide 76 (e.g., a sapphire block) at a distal end of a dermatological handpiece. As in previous embodiments, the light waveguide 76 extends from a proximal end 76a to a distal end 76b, which is adapted for contact with the skin. A pressure applicator 74, which surrounds the waveguide 76, includes a channel 78, formed between a sidewall 76c of the waveguide 76 and an inner wall 80 of the applicator's housing, that has a proximal opening 78a for coupling with a source of pressure (e.g., positive pressure in this case) and a distal end via which a pressure (e.g., positive pressure in this case) can be applied to a skin portion 84 on the periphery of a skin target 86, which is in contact with the distal end of the waveguide, so as to generate a positive pressure zone.

The pressure applicator 74 further includes another channel 88, formed between the inner wall 80 and an outer wall 82, for applying an opposite pressure (e.g., a negative pressure in this case) to a skin portion 90 that is farther from the skin target that the portion to which a positive pressure is applied. More specifically, the channel 88 includes a proximal opening 88a for coupling to a source of pressure (e.g., negative pressure in this case) and a distal opening 88b via which a negative pressure can be applied to the skin to generate a negative pressure zone. In the case of the negative pressure zone, a pressure seal between the applicator's housing and the skin can be generated via a normal force applied by the distal tips of the outer wall 82 and the inner wall 80 against the skin. Similarly, the normal force of the distal tip of the light waveguide and that of the inner wall 80 against the skin can generate a pressure seal for the positive pressure zone. In some applications, the positive and negative pressure zones can be interchanged, e.g., by applying a positive pressure to the channel 88 and a negative pressure to the channel 78. In some cases, by adjusting the positive and negative pressures, the skin target can be maintained in tension while in contact with the distal end 76b of the light waveguide 76. The applied negative pressure can be in a range of about 2 inch Hg to about 30 inch Hg, preferably in the range of about 5 inch Hg to about 20 inch Hg, or more preferably in the range of about 7 inch Hg to about 12 inch Hg.

Positive pressure may be applied in a wide range from, for example, 0 to 200 inches Hg., although higher pressures are potentially feasible. In embodiments such as those shown in FIGS. 1A-3B, the amount of positive pressure will be limited by the amount that the operator can effectively resist when placing the device against the tissue, for example, approximately 6 inches of Hg in some embodiments. However, in alternate embodiments, a band or other mechanism can be employed to secure the device during operation such that much higher pressures can be applied. For example, in an embodiment designed to treat tissue of the thigh, a band or strap can be fastened around the leg and secured to hold the device in place.

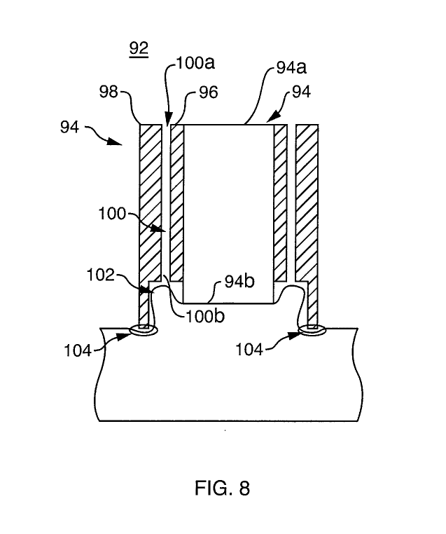

FIG. 8 schematically depicts a pressure applicator 92 according to another embodiment of the invention that is coupled to a distal end of a dermatological handpiece surrounding a light waveguide 94 (e.g., a sapphire block) thereof. Similar to the previous embodiments, the light waveguide 94 includes a proximal end 94a that extends to a distal end 94b that is adapted for contact with the skin. The pressure applicator 92 includes a telescopic housing 94 formed of an inner wall 96 and an outer wall 98 between which a channel 100 is formed, which extends from a proximal end 100a to a distal end 10b. The distal end 100b of the channel 100 opens into a pressure (e.g., negative pressure) cavity 102. In this embodiment, the distal end of the channel is offset (set back) relative to the light waveguide's distal end. This offset can be, e.g., in a range of greater than 0 mm to about 60 mm, although other offsets are possible. The preferred offset will vary depending on the application, with a lesser offset for thinner tissues, such as skin around the chin and eyes and a greater offset for softer or thicker tissues such as tissue containing a large amount of fat and/or cellulite.

In some embodiments, negative pressure is applied only to the periphery of the target region while positive pressure is applied to the target region. For example, the optical element can extend beyond the pressure applicator and negative pressure is applied around the periphery of the optical element through the channel in the applicator. The pressure applicator 92 further includes one or more inflatable cuff(s) 104 that are coupled to a distal end of the applicator's telescopic housing.

With continued reference to FIG. 8, in use, the skin beneath the waveguide 94 (skin target) is held in tension by a combination of a normal force exerted against the skin by the initially deflated cuff(s) and an opposing force on the skin generated by a negative pressure in the cavity 102. Further, the cuff(s) can be inflated so as to gather more skin into the negative pressure cavity 102. This can cause further deformation of the skin surface so as to increase the tension in the skin target. The cuffs can be inflated using a separate pressure source or the same pressure source as is used to generate positive pressure, using an appropriate valve system.

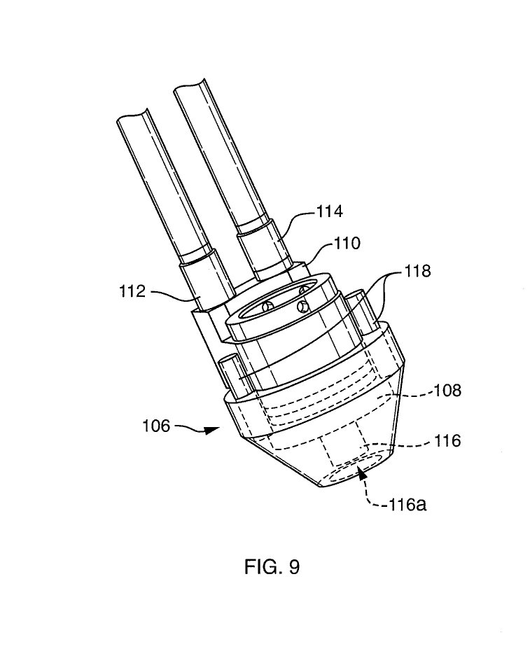

By way of further illustration, FIG. 9 schematically depicts an exemplary pressure applicator 106 according to one embodiment of the invention that is designed to be coupled to a distal portion of a handpiece, surrounding a radiation waveguide 108, e.g., a sapphire block, which is cooled by a cooling plate 110. The plate 110 is, in turn, cooled by the flow of a cooling fluid (such as water) through one or more inner passages (not shown) thereof. The cooling is sufficient to cool the surface of the radiation waveguide so that the epidermis is cooled when in contact with the surface of the radiation waveguide. For example, the cooling fluid can be introduced into the cooling plate via an input port 112 and can exit via an output port 114, thereby removing heat from the plate. The pressure applicator 106 includes a vacuum chamber 116 that is in fluid communication with vacuum connections 118, which are adapted for coupling to a negative pressure source (not shown)—though in other implementations they can be coupled to a positive pressure source. Further, the vacuum chamber 116 includes an opening 116a at a distal end thereof through which a negative pressure can be applied to the skin.

In use, the distal end of the vacuum chamber can be placed into contact with the skin so as to form a seal around a skin portion encompassed by the opening 116a. Upon application of a negative pressure, the skin portion is drawn into the vacuum chamber. As the negative pressure is maintained, the skin portion continues to be lifted towards the distal surface of the waveguide until it is in contact therewith. In many embodiments, the applied negative pressure (e.g., in a range of about 2 inch Hg (6.7×10³ Pa) to about 30 inch Hg (1×10⁵ Pa), preferably in the range of about 5 inch Hg (16.9×10³ Pa) to about 20 inch Hg (67.7×10⁵ Pa), or more preferably in the range of about 7 inch Hg (23×10³ Pa) to about 12 inch Hg (41×10³ Pa)) can continue to stretch the skin portion such that it would cover the distal surface of the waveguide.

With continued reference to FIG. 9, in some embodiments, the pressure applicator 106 can be removably and replaceably coupled, via a pressure fit, to the distal end of the handpiece. By way of example, the applicator can be formed of a polymeric material that can be readily removed and attached to the handpiece.

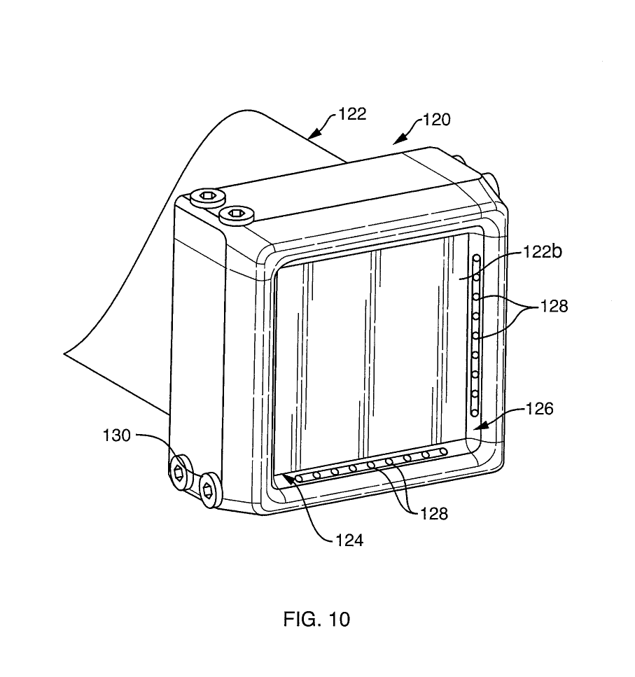

By way of another example, FIG. 10 schematically depicts a pressure applicator assembly 120 that is coupled to an EMR waveguide 122 of a handpiece according to an embodiment of the invention. A recessed surface 122b of the waveguide, through which radiation is applied to the skin, forms top surface of a recessed cavity 124 whose sidewalls (e.g., sidewall 126) include a plurality of vacuum inlet ports 128. The inlet ports 128 are in fluid communication with a pair of vacuum connections 130, which can in turn be coupled to a source of negative pressure. While in some implementations, the sidewalls of the vacuum chamber are substantially perpendicular to the recessed surface 122, in others, they can be slanted away from that surface.

In use, the pressure applicator can be placed over the skin such that the distal tips of its sidewalls would form a seal around a skin portion. In this manner, the recessed cavity 124 can be turned into a chamber to which a negative pressure—or a positive pressure in other implementations—can be applied via the inlet ports 128. The applied negative pressure can draw the skin portion into the chamber. In many embodiments, the level of the negative pressure is such that the skin portion is lifted to be in contact with the recessed surface 122b of the waveguide. Further, the negative pressure applied to the skin portion by the inlet ports causes tension therein in a direction substantially parallel to the waveguide's surface 122.

In other embodiments, the skin can be stretched in different axial directions. For example, negative pressure can be applied only to two opposing sides of the pressure applicator shown in FIG. 10. Then, the negative pressure can be turned off from those two sides and turned on at the remaining two opposing sides. Different patterns of skin stretching can be achieved by controlling the negative pressure applied to the skin.

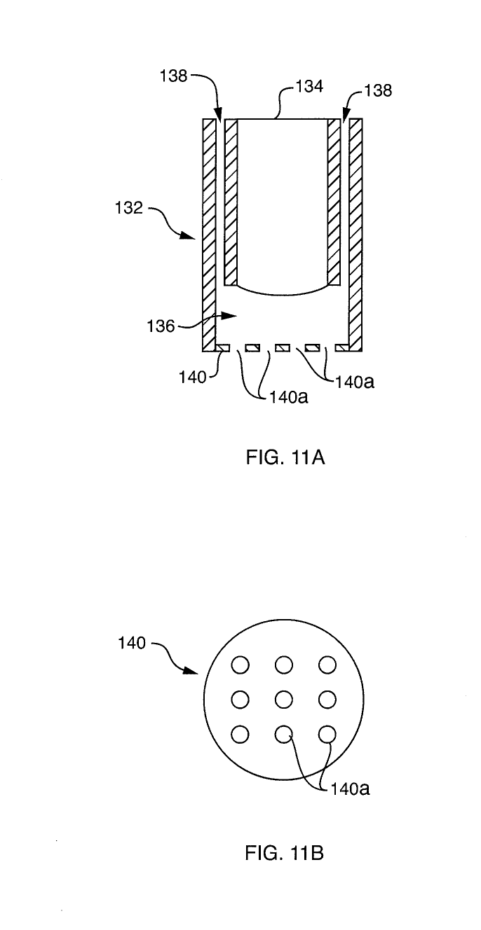

In some embodiments, a pressure applicator of the invention allows applying positive and/or negative pressure to a plurality of locations within a skin target so as to cause redistribution of the volumetric blood content within the target. By way of example, FIGS. 11A-11B schematically depicts such a pressure applicator 132 that is adapted for coupling to a radiation waveguide 134 of a dermatological handpiece. The applicator 132 includes a pressurization chamber 136 to which a positive or a negative pressure can be applied via a plurality of passages 138 by a source of pressure (not shown), e.g., a vacuum pump. A radiation-transmissive mask 140 (e.g., formed from a polymeric material transparent to radiation to be applied to the skin) having a plurality of openings 140a, which forms a bottom surface of the chamber 136, is adapted for contact with the skin (FIG. 11B provides a schematic top view of the mask).

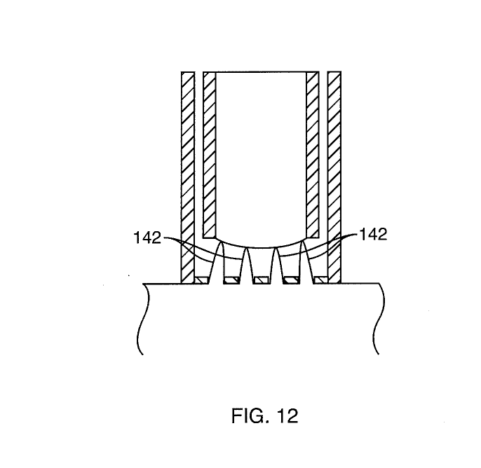

In use, the pressure mask can be placed against a skin target and a positive or negative pressure can be applied to the chamber, and consequently through the openings 140a to a plurality of skin segments lying beneath those openings. By way of example, with reference to FIGS. 11A and 12, upon application of a sufficiently strong negative pressure to the chamber 136 (e.g., a negative pressure in a range of about 2 inch Hg to about 30 inch Hg, preferably in the range of about 5 inch Hg to about 20 inch Hg, or more preferably in the range of about 7 inch Hg to about 12 inch Hg), a plurality of skin segments 142 lying under the openings 140a can be drawn into the chamber causing tension in the skin segments lying beneath the solid portions of the pressure mask, which are under positive pressure as the mask is pressed against the skin. This can cause a redistribution of blood volume between within the skin target characterized by an increase in the volumetric blood content in the segments that are under negative pressure and a respective decrease in the blood content of those segments that are under positive pressure.

Such a redistribution of blood can be useful since it allows different types of treatment during a single radiation exposure. For example, treatment requiring absorption by blood (e.g., treatment of vascular lesions) can be delivered to skin segments exposed to negative pressure. Treatment requiring absorption by tissue targets at depth (e.g., acne) or absorption by chromophores that substantially overlap with skin (such as melanin for hair removal) can be delivered to the area surrounding the skin segments that are exposed to the negative pressure. Positive pressure can be applied to the surround skin to enhance absorption.

Suitable Light Sources

Optical system 10 includes an EMR source 12, which source can be a coherent light source, such as a solid-state laser, dye laser, diode laser, fiber laser or other coherent light source, or can be an incoherent light source, for example a flash lamp, halogen lamp, light bulb or other incoherent light source used to deliver optical radiation in dermatology procedures. Acoustic, RF or other EMR sources may also be employed in suitable applications. The output from source 12 is applied to an optical element 28, which is drawn in contact with the surface of the patient's skin upon application of pressure as shown in FIGS. 1B and 2B. Where an acoustic, RF or other EMR source is used as source 12 system 10 would be a suitable system for concentrating or focusing such EMR, for example a phased array, and the term “optical system” should be interpreted, where appropriate, to include such system.

Examples of Solid State Light Sources Include:

1. Light Emitting Diodes (LEDs): these include edge emitting LED (EELED), surface emitting LED (SELED) or high brightness LED (HBLED). The LED can be based on different materials, such as, without limitation, GaN, AlGaN, InGaN, AlInGaN, AlInGaN/AlN, AlInGaN (emitting from 285 nm to 550 nm), GaP, GaP:N, GaAsP, GaAsP:N, AlGaInP (emitting from 550 nm to 660 nm) SiC, GaAs, AlGaAs, BaN, InBaN, (emitting in near infrared and infrared). Another suitable type of LED is an organic LED using polymer as the active material and having a broad spectrum of emission with very low cost.

2. Superluminescent diodes (SLDs): An SLD can be used as a broad emission spectrum source.

3. Laser diodes (LD): A laser diode can be an effective light source (LS). A wave-guide laser diode (WGLD) is very effective but is not optimal due to the difficulty of coupling light into a fiber. A vertical cavity surface emitting laser (VCSEL) may be most effective for fiber coupling for a large area matrix of emitters built on a wafer or other substrate. This can be both energy and cost effective. The same materials used for LED's can be used for diode lasers.

4. Fiber laser (FL) with laser diode pumping.

5. Fluorescence solid-state light source with electric pumping or light pumping from LD, LED or current/voltage sources (FLS). An FLS can be an organic fiber with electrical pumping.

6. For a description of additional useful light sources and various techniques, including wavelength, that can be utilized to control the depth to which radiation is concentrated and suitable optical systems that can be used to concentrate applied radiation in parallel or in series for selected combinations of one or more treatment portions

Other suitable low power lasers, mini-lamps or other low power lamps or the like may also be used as light source(s) in embodiments of the present invention.

LED's are the currently preferred radiation source because of their low cost, the fact that they are easily packaged, and their availability at a wide range of wavelengths suitable for treating various tissue conditions. In particular, Modified Chemical Vapor Deposition (MCVD) technology may be used to grow a wafer containing a desired array, preferably a two-dimensional array, of LED's and/or VCSEL at relatively low cost. Solid-state light sources are preferable for monochromatic applications. However, a lamp, for example an incandescent lamp, fluorescent lamp, micro halide lamp or other suitable lamp is a preferable light source for applying white, red, near infrared, and infrared irradiation during treatment.

Since the efficiency of solid-state light sources is 1-50%, and the sources are mounted in very high-density packaging, heat removal from the emitting area is generally the main limitation on source power. For better cooling, a matrix of LEDs or other light sources can be mounted on a diamond, sapphire, BeO, Cu, Ag, Al, heat pipe, or other suitable heat conductor. The light sources used for a particular apparatus can be built or formed as a package containing a number of elementary components. For improved delivery of light to skin from a semiconductor emitting structure, the space between the structure and the skin can be filled by a transparent material with a refractive index in the range 1.3 to 1.8, preferably between 1.35 and 1.65, without air gaps. In an alternate embodiment, a material, e.g., a gel having such an index, can be inserted into the vacuum chamber to fill the space between the tissue and the optical window when negative pressure is applied.

The invention can use a low-power optical radiation source, or preferably an array of low power optical radiation sources, in a suitable head which is either held over a treatment area for a substantial period of time, e.g. one second to one hour, or is moved over the treatment area a number of times during each treatment. Depending on the area of the person's body and the condition being treated, the cumulative dwell time over an area during a treatment will vary. The treatments may be repeated at frequent intervals, e.g. daily, or even several times a day, weekly, monthly or at other appropriate intervals. The interval between treatments may be substantially fixed or may be on an “as required” basis. For example, the treatments may be on a substantially regular or fixed basis to initially treat a condition, and then be on as an “as required” basis for maintenance. Treatment can be continued for several weeks, months, years and/or can be incorporated into a user's regular routine hygiene practices. Certain treatments are discussed further in U.S. application Ser. No. 10/740,907, entitled “Light Treatments For Acne And Other Disorders Of Follicles,” filed Dec. 19, 2003, which is incorporated herein by reference in its entirety.

Thus, while light has been used in the past to treat various conditions, such treatment has typically involved one to ten treatments repeated at widely spaced intervals, for example, weekly, monthly or longer. By contrast, the number of treatments for use with embodiments according to aspects of this invention can be from ten to several thousand, with intervals between treatments from several hours to one week or more. It is thought that, for certain conditions such as acne or wrinkles, multiple treatments with low power could provide the same effect as one treatment with high power. The mechanism of treatment can include photochemical, photo-thermal, photoreceptor, photo control of cellular interaction or some combination of these effects. For multiple systematic treatments, a small dose of light can be effective to adjust cell, organ or body functions in the same way as systematically using medicine.

Instead of using single or few treatments of intense light, which must be performed in a supervised condition such as a medical office, the same reduction of the bacteria population level can be reached using a greater number of treatments of significantly lower power and dose using, for example, a hand-held photocosmetic device in the home. Using a relatively lower power treatment, a consumer can use the photocosmetic device in the home or other non-medical environment.

The specific light parameters and formulas of assisted compounds suggested in the present invention provide this treatment strategy. These treatments may preferably be done at home, because of the high number of treatments and the frequent basis on which they must be administered, for example daily to weekly. Some embodiments of the present invention could additionally be used for therapeutic, instructional or other purposes in medical environments, such as by physicians, nurses, physician's assistants, physical therapists, occupational therapists, etc.

Depending on the treatment to be performed, the light source may be configured to emit at a single wavelength, multiple wavelengths, or in one or more wavelength bands. The light source may be a coherent light source, for example a ruby, alexandrite or other solid state laser, gas laser, diode laser bar, or other suitable laser light source. Alternatively, the source may be an incoherent light source for example, an LED, arc lamp, flash lamp, fluorescent lamp, halogen lamp, halide lamp or other suitable lamp.

Various light based devices can be used to deliver the required light doses to a body. The optical radiation source(s) utilized may provide a power density at the user's skin surface of from approximately 1 mwatt/cm² to approximately 100 watts/cm², with a range of 10 mwatts/cm² to 10 watts/cm² being preferred. The power density employed will be such that a significant therapeutic effect can be achieved by relatively frequent treatments over an extended time period. The power density will also vary as a function of a number of factors including, but not limited to, the condition being treated, the wavelength or wavelengths employed and the body location where treatment is desired, i.e., the depth of treatment, the user's skin type, etc. A suitable source may, for example, provide a power of approximately 1-100 watts, preferably 2-10 W.

Additionally, in alternative embodiments, depending on the desired treatment, different wavelengths of light will enhance the effect. For example, when treating acne, a wavelength band from 290 nm to 700 nm is generally acceptable with the wavelength band of 400-430 nm being preferred as described above. For the stimulation of collagen, the target area for this treatment is generally the papillary dermis at a depth of approximately 0.1 mm to 0.5 mm into the skin, and since water in tissue is the primary chromophore for this treatment, the wavelength from the radiation source should be in a range highly absorbed by water or lipids or proteins so that few photons pass beyond the papillary dermis. A wavelength band from 900 nm to 20000 nm meets these criteria. For sebaceous gland treatment, the wavelength can be in the range 900-1850 nm, preferable around peaks of lipid absorption as 915 nm, 1208 nm, and 1715 nm. Hair growth management can be achieved by acting on the hair follicle matrix to accelerate transitions or otherwise control the growth state of the hair, thereby accelerating or retarding hair growth, depending on the applied energy and other factors, preferable wavelengths are in the range of 600-1200 nm.

In alternative embodiments, the light source may generate outputs at a single wavelength or may generate outputs over a selected range of wavelengths or one or more separate bands of wavelengths. Light having wavelengths in other ranges can be employed either alone, or in conjunction with other ranges, such as the 400-430 nm to take advantage of the properties of light in various ranges. For example, light having a wavelength in the range of 480-510 nm has anti-bacterial properties, but also is less effective in killing bacteria than light having wavelengths in the range of 400-430 nm. However, light having a wavelength in the range of 480-510 nm also is known to penetrate relatively deeper into the porphyrins of the skin than light in the range of 400-430 mm.

Similarly, light having a wavelength in the range of 550-600 nm is known to have anti-inflammatory effects. Thus, light at these wavelengths can be used alone in a device designed to reduce and/or relieve inflammation and swelling of tissue (e.g., inflammation associated with acne). Furthermore, light at these wavelengths can be used in combination with the light having the wavelengths discussed above in a device designed to take advantage of the characteristics and effects of each range of wavelengths selected.

In embodiments of a photocosmetic device capable of treating tissue with light of multiple wavelengths, multiple light sources could be used in a single device, to provide light at the various desired wavelengths, or one or more broad band sources could be used with appropriate filtering. Where a radiation source array is employed, each of several sources may operate at selected different wavelengths or wavelength bands (or may be filtered to provide different bands), where the wavelength(s) and/or wavelength band(s) provided depend on the condition being treated and the treatment protocol being employed. Similarly, one or more broadband sources could be used. For a broadband source, filtering may be required to limit the output to desired wavelength bands. An LED module could also be used in which LED dies that emit light at two or more different wavelengths are mounted on a single substrate and electrically connected to all the various dies to be controlled in a manner suitable for the treatment for which the device is designed, e.g., controlling some or all of the LED dies at one wavelength independently or in combination with LED dies that emit light at other wavelengths.

Employing sources at different wavelengths may permit concurrent treatment for a condition at different depths in the skin, or may even permit two or more conditions to be treated during a single treatment or in multiple treatments by selecting a different mode of operation of a photocosmetic device. The depth of treatment can be controlled through the use of the pressure controller so that absorption of radiation is increased at the target depth. Examples of wavelength ranges for various treatments are provided in the Table 1 below:

TABLE 1
Examples of wavelength ranges useful for the treatment of specific diseases and
cosmetic conditions.

| Treatment condition or application | Wavelength, nm |
|---|---|
| Acne | 290-700, 900-1850 |
| ALA lotion with PDT effect on skin | 290-700 |
| condition including anti cancer effect | |
| Alopecia | 620-680 and 760-880 nm |
| Anti-aging | 400-2700 |
| Blood, lymph, immune system | 290-1350 |
| Burns | 760-880 nm |
| Cellulite | 600-1350 |
| Color lotion delivery into the skin | Spectrum of absorption of color center and |
| | 1200-20000 |
| Deep vascular | 500-1300 |
| Deep wrinkle, elasticity | 500-1350 |
| Direct singlet oxygen generation | 1260-1280 |
| Gingivitis | 380-450 and 600-700 nm |
| Gum inflammation | 380-450 and 600-700 nm |
| Hair growth control | 380-1350 |
| Lentigo senile | 600-700 nm |
| Lotion delivery into the skin | 1200-20000 |
| Lotion with PDT effect on skin condition | Spectrum of absorption of photo sensitizer |
| including anti cancer effect | |
| Muscular, joint treatment | 600-1350 |
| Odor | 290-1350 |
| Oiliness | 290-700, 900-1850 |
| Pain relief | 500-1350 |
| Pseudofolliculitis barbae (PFB) | 300-400, 450-1200 |
| Pigmented lesion, de pigmentation | 290-1300 |
| Psoriasis | 290-700 |
| Scars | 380-420, 620-680 and 760-830 nm |
| | (depending on scar nature) |
| Skin cleaning | 290-700 |
| Skin lifting | 600-1350 |
| Skin rejuvenation | 600-700 and 760-880 nm |
| Skin texture, stretch mark, scar, porous | 290-2700 |
| Striae | 760-880 nm |
| Superficial vascular | 290-600 |
| | 1300-2700 |
| Wrinkles | 620-680 and 760-880 nm |
| Wound healing | 380-1250 nm (depending on wound nature) |

Providing negative pressure to apply tension to the target area also may enhance treatment to tissues at a depth.

The applied radiation preferably has an output wavelength which is at least in part a function of the at least one depth of the treatment portions. More specifically, the wavelength of the applied radiation may be selected as a function of the applied radiation as follows: depth=0.05 to 0.2 mm, wavelength=400-1880 nm & 2050-2350 nm, with 800-1850 nm & 2100-2300 nm preferred; depth=0.2 to 0.3 mm, wavelength=500-1880 nm & 2050-2350 nm, with 800-1850 nm & 2150-2300 nm preferred; depth=0.3 to 0.5 mm, wavelength=600-1380 nm & 1520-1850 nm & 2150-2260 nm, with 900-1300 nm & 1550-1820 nm & 2150-2250 nm preferred; depth=0.5 to 1.0 mm, wavelength=600-1370 nm & 1600-1820 nm, with 900-1250 nm & 1650-1750 nm preferred; depth=1.0 to 2.0 mm, wavelength=670-1350 nm & 1650-1780 nm, with 900-1230 nm preferred; depth=2.0 to 5.0 mm, wavelength=800-1300 nm, with 1050-1220 nm preferred. In addition, table 1 shows a chart of optimal light wavelengths for vascular treatment at various depths. For further discussion of treatment parameters see U.S. Pat. No. 6,997,923, issued Feb. 14, 2006 entitled “Method and Apparatus for EMR Treatment,” and U.S. application Ser. No. 10/331,134 filed Dec. 27, 2002 entitled “Method And Apparatus For Improved Vascular Related Treatment,” which is incorporated by reference in its entirety.

Exemplary Uses

The apparatus and methods can be used for a variety of dermatological treatments, such as, but not limited to, acne treatment, removal of unwanted hair, skin rejuvenation, removal of vascular lesions, treatment of vascular lesions, pigmented lesions, port wine stains, psoriasis, scar, or other skin blemishes, treatment of cellulite, pigmented lesions and psoriasis, tattoo removal, treatment of skin and other cancers, etc. The methods can be performed on any body component on which optical dermatology procedures are performed. In particular, the methods are particularly suited to procedures on areas of the body containing loose skin, such as the abdomen, thighs, arms, cheeks, and buttocks.

In some embodiments, the application of negative pressure to bring the target region in contact with the distal surface of the optical element, as provided by the present invention, makes the skin at the target region thinner which enhances treatment by changing the thickness of the basal membrane. The basal membrane, which consists of tissue with high melanin, can be a barrier to efficiently passing an effective amount of radiation to targets below the skin surface. Through compression, optical density and light absorption of basal layer is reduced which increases deeper light penetration. In addition, compression brings deeper tissue structures closer to the light source. Stretching of the skin in the treatment area also allows more efficient cooling by reducing the conduction path from the chilled waveguide to the heat sensitive elements of the skin. When skin at the target region is held in tension, blood flow is restricted thereby minimizing heat flux due to blood perfusion. This allows increased energy delivery while maintaining patient comfort as well as safety.

Specific exemplary applications are described below, but other embodiments may be used for other applications as well.

Skin Rejuvenation

In one exemplary use, an embodiment is adapted for skin rejuvenation treatments by collagen regeneration. In such treatments, since collagen is not itself a chromophore, a chromophore such as water in the tissues or blood in the papillary dermis or below typically absorbs radiation and is heated to heat the adjacent collagen, causing selective damage or destruction thereof which results in collagen regeneration. Perturbing blood vessels in the region can also result in the release of fibroblasts which trigger the generation of new collagen. The methods and apparatus of the invention can enhance skin rejuvenation. The pressure applied to the skin can be modulated to enhance collagen regeneration and enable radiation to pass through the skin to the appropriate target.

Treatments can be made along the line of a wrinkle or other blemish to be treated. Such procedures can be performed over a relatively large area or can be performed by periodically firing a beam when over a wrinkle, the beam being traced in a predetermined pattern and fired only when over selected points on the wrinkle, or being moved to track a wrinkle and periodically fired while thereover. Also, as for other treatments where the teachings of this invention are employed, healing occurs relatively quickly so that a subsequent treatment, to the extent required, might generally be performed within a few weeks of an initial treatment, and certainly in less than a month. Typically, a bump in the skin occurs when collagen is heated, the bump resulting from contraction of the collagen. Thus, this technique can be used not only to remove wrinkles but also to remove other skin blemishes such as acne or chicken pox scars or other scars in the skin and may also be utilized for treating cellulite.

Other skin blemishes treatable by the teachings of this invention include stretch marks, which differ from wrinkles in that these marks are substantially flush with the surface. Hypotropic scarring, the raised scars which occur after surgery or certain wounds, can also be treated by reducing blood flow to the vessels of the scar in much the same way that port wine stains are treated above.

In some embodiments, fractional treatment methods can be enhanced through the use of the methods of the present invention. For example, the embodiment shown in FIG. 9 is a head that can be attached to the Lux1540™ Fractional handpiece manufactured by Palomar Medical Technologies, Inc., which can be used, for example, for soft tissue coagulation and non-ablative skin resurfacing. Additional, embodiments employing fractional technology can be used to perform, without limitation, hair removal, treatment of vascular lesions, treatment of sebaceous glands, for example to treat acne, treatment of subcutaneous fat, treatment of cellulite, and skin resurfacing on areas where such treatments cannot currently be performed, for example neck and hands, where the damage caused using standard skin resurfacing techniques does not normally heal. The treating of only small areas surrounded by areas of untreated tissue provides healthy tissue that causes the treated tissue to heal more quickly.

Embodiments of the present invention can also be used for vascular treatments, such as the treatment of vascular lesions and varicose veins. As discussed above, depths of treatment can be optimized and controlled by the selection of parameters such as wavelength and power density. The additional use of positive and negative pressure can further augment the treatment and improve efficacy. Wavelength parameters for such treatments are outlined below in Table 2.

TABLE 2
Optimal light wavelengths for vascular treatment.

| | | | Skin | Skin | Skin | Skin | Skin | Skin |
|---|---|---|---|---|---|---|---|---|
| Vessel | | | type I | type II | type III | type IV | type V | type VI |
| | Depth, | Diameter, | Spectra, | Spectra, | Spectra, | Spectra, | Spectra, | Spectra, |
| Type | mm | mm | nm | nm | nm | nm | nm | nm |
| Plexus | 0.1 | 0.01 | 400-430 | 405-435 | 405-435 | 410-440 | 410-440 | 410-440 |
| Superficial | 0.25 | 0.25 | 410-440 | 415-445 | 415-445 | 415-445 | 420-450 | 420-450 |
| | | | 510-595 | 510-595 | 510-595 | 510-595 | 540-595 | 540-595 |
| Intermediate | 0.5 | 0.5 | 510-600 | 510-600 | 530-600 | 530-600 | 530-600 | 530-600 |
| Deep | >1 | >1 | 510-600 | 510-600 | 530-600 | 530-600 | 900-1100 | 900-1100 |
| | | | 800-1100 | 800-1100 | 800-1100 | 850-1100 | | |

The teachings of this invention may, as indicated above, also be utilized for tattoo removal, for treating pigmented lesions, for treating hypotropic and other scars, stretch marks, acne and chicken pox scars and other skin blemishes and for treating various other conditions which may exist in the patient's body at depths, for example, various skin cancers and possibly PFB. Examples of wavelength ranges useful for the treatment of specific diseases and cosmetic conditions is contained in Table 1 above. The negative pressure applied at the treatment area can be adjusted to maximize treatment at depth. For skin tumors, a combination may be used of a feedback system that localizes the position of the tumor, controls the pressure to maximize absorption of radiation at the tumor site, and a robotic system that insures complete thermal destruction of the tumor. Psoriasis may be treated in substantially the same way with substantially the same parameters as for port wine stain. The teachings may also be used to treat intradermal parasites such as larva migrans, which can be detected and selectively killed using the teachings of the invention.

Acne

An example of a condition that is treatable using an embodiment of the present invention is acne. In one aspect, the treatment described involves the destruction of the bacteria Propionibacterium Acnes (P. acnes) responsible for the characteristic inflammation associated with acne. Destruction of the bacteria may be achieved by targeting porphyrins stored in P. Acnes. Porphyrines, such as protoporphyrins, coproporphyrins, and Zn-protoporphyrins are synthesized by anaerobic bacteria as their metabolic product. Porphyrines absorb light in the visible spectral region from 400-700 nm, with strongest peak of absorption in the range of 400-430 nm. By providing light in the selected wavelength ranges in sufficient intensity, photodynamic process is induced that leads to irreparable damage to structural components of bacterial cells and, eventually, to their death. In addition, heat resulting from absorption of optical energy can accelerate death of the bacteria. For example, the desired effect can be achieved using a light source emitting light at a wavelength of approximately 405 nm using an optical system designed to irradiate tissue 0.2-1 mm beneath the skin surface at a power density of approximately 0.01-10 W/cm² at the skin surface. Blue light (400 to 450 nm), which is most effectively absorbed by porphyrins, has very limited penetration depth in normal blood-containing skin. More precisely, the penetration depth of such light does not exceed ˜300 μm, whereas the population density of P. Acnes (primary target of the PDT) peaks at ˜1.2 mm depth.

Thus, through the methods of the present invention, more effective acne treatment can be achieved. For example, negative pressure in a range of about 2 inch Hg to about 30 inch Hg, preferably in the range of about 5 inch Hg to about 20 inch Hg, or more preferably in the range of about 7 inch Hg to about 12 inch Hg. can be applied to a skin region in order to draw a portion of the skin region (e.g., the skin target) into contact with the optical surface of an optical element so as to reduce tissue inhomogeneity, expel blood from skin vessels, and/or reduce travel distance of the radiation to the treatment region, thereby increasing penetration depth of the applied radiation. Description of treatments that can be enhanced with the use apparatus and methods of the present invention are discussed in U.S. application Ser. No. 10/740,907, entitled “Light Treatments For Acne And Other Disorders Of Follicles,” filed Dec. 19, 2003, which is incorporated herein by reference in its entirety.

In another aspect of the invention, the treatment can cause resolution or improvement in appearance of acne lesion indirectly, through absorption of light by blood and other endogenous tissue chromophores. The amount of negative pressure can be varied to deliver radiation to the desired tissue at the desired depth. In some embodiments, treatment of the tissue surrounding the lesion and the lesion can be simultaneously treated. For example, a waveguide with a plurality of holes can be used as described in FIGS. 3A&B and 11A&B.

Tattoo Removal and Pigmented Lesions

In some embodiments, the invention can be used to treat birthmarks or other pigmented lesions in the epidermis. Such lesions are generally difficult to treat without blistering using conventional treatment. The stretching of the skin thins the basal layer which enhances the absorption of the treatment radiation. For example, 4×-5× more energy can be transmitted to the target through the application of negative pressure resulting in skin stretching.

Port wine stains and tattoos require treatment at a greater depth. Thus, the negative pressure would be increased so that the treatment area can be effectively targeted. In all cases, a first treatment might result in only the lightening of the treated area. Once the treated portion has healed, which generally would occur in a few weeks to a month, one or more additional treatments can be performed to further lighten the treated area until the lesion, port wine stain, tattoo or the like is removed. In each instance, dead cells resulting from the treatment containing melanosites, ink or the like, would be removed by the body, normally passing through the lymphatic system.

There are three general ways in which the invention may be utilized for tattoo removal. The first is by using a wavelength or wavelengths absorbed by the tattoo ink, preferably with short, high fluence pulses, to break up or destroy the ink in and between cells. The second technique involves destroying the cells containing the ink, targeting either the ink or water in the cells, causing the ink to be released and removed by the body's lymphatic system. Here long pulses in the millisecond to second range, having low power and high energy, would typically be utilized. In a third technique, an ablation laser would be used to drill 1 to 2 mm spots into the tattoo, ablating or vaporizing both cells and tattoo ink in these areas. A randomized pattern on each treatment is also preferable to interference of the removal pattern.

Fat Reduction

In some embodiments, the methods and apparatus can be used to reduce lipid-rich or fatty tissue. By applying negative pressure to loose skin or fatty tissue, the radiation can be absorbed more readily by the fat cells. While compression of skin in a bony area, such as arms, legs, and shoulders can be easily accomplished through positive pressure applied to the photocosmetic device, areas with loose skin, such as the abdomen and thighs are more difficult to adequately compress. By lifting the skin through the application of negative pressure, uncompensated internal blood pressure inside the skin opens up blood vessels which increases blood volume content of the skin. When skin is brought in contact with the light guide distal tip, positive pressure is then applied to the skin surface which redistributes blood from the skin vessels beneath the distal tip to unconstrained portion of skin, which increases skin transparency for certain wavelength and improves the therapeutic effect of treatment due to deeper light penetration. Where subcutaneous fat is being non-invasively treated, duration of radiation pulse and the temperature to which the fat or lipid tissue is heated are critical to the desired results. For example, at increased temperature, fat is altered by a biochemical reaction or lipolysis, while for higher temperatures and sufficient pulse duration, fat cells are killed, permitting the cells and liquid lipid therein to be absorbed. At still higher temperatures, cell membranes are destroyed, permitting lipid pools to be formed. These pools may also be absorbed but, since free fatty acid in lipid can be toxic in sufficient quantity, if substantial quantities of fat cell membranes have been destroyed, permitting a large lipid pool to be formed, it is preferable to remove the lipid, for example with a cannula or needle. The heated collagen of supporting structure may react to provide a more pleasing skin appearance after treatment and avoid sagging folds of skin or skin depressions where the lipid tissue has been destroyed. While all of the fat in a subcutaneous layer may be treated, it is difficult to get sufficient energy deep into the fat, so treatment has generally been restricted to a surface layer of the fat. Through the use of the methods and apparatus of the present invention, sufficient energy can penetrate deep into the fat, thereby improving the therapeutic while minimizing treatment sessions. Repetitive treatments can be performed to remove successive layers of the subcutaneous fat.

Where lipid-rich tissue/fat surrounds a vessel, organ or other anatomical element, the irradiation can be performed through an optical element which is brought in contact with the skin surface above the fat to be treated. Using the apparatus and methods of this invention, reduces the amount of water rich tissue that the radiation must pass through to reach the fat. Therefore, better absorption of the appropriate wavelengths by the target subcutaneous fat is possible, which improves the efficiency of the procedure. For description of radiation protocols see U.S. Pat. No. 7,060,061, issued Jun. 13, 2006 and U.S. Pat. No. 6,605,080 issued Aug. 12, 2003, which are incorporated herein in their entirety.

Hair Removal

Maintaining the optical element in good thermal and optical contact with the surface of the patient's skin while applying radiation from the source, whether located external to head or within the head, offers a number of significant advantages when performing various dermatological treatments. The optical element being in good optical contact with the patient's skin improves the efficiencies of energy transfer into the skin, reducing the size and cost of the required energy source. Further, the optical element being in good thermal contact with the patient's skin permits the optical element to be used to heat the volume in the patient's dermis at which treatment is to occur, for example the area of bulb for a hair removal procedure, so as to reduce the amount of energy required from the radiation source in order to perform the desired procedure at this volume, thus further reducing the cost of such source. Good thermal contact also permits the head to be utilized to cool the patient's epidermis before irradiation, during irradiation, and after irradiation, to protect the epidermis from thermal damage. Applying pressure to optical element stretches the skin in the treatment area which can provide a number of advantages, including reducing the physical distance between the head and the target volume, reducing the coefficient of scattering in the skin so that more of the applied radiation reaches the target volume and, for hair removal, flattening the hair follicle so as to increase the area of the follicle exposed to radiation. All of these effects reduce the amount of radiation required from the source, thereby further reducing the cost of the system. Various techniques are available for measuring/detecting good thermal contact between a head and the patient's skin including the temperature profile detecting technique of U.S. Pat. No. 6,273,884 issued Aug. 14, 2001 entitled “Method and Apparatus for Dermatology Treatment,” which is incorporated herein by reference. FIGS. 9 and 10 illustrate exemplary embodiments for pressure applicators suitable for use in practicing the teachings of this invention.

Exemplary parameters for hair removal are: Wavelength: 600-1200 nm; average power per length unit: 5-150 W/cm; width of beam along direction of scanning: 0.05-5 mm; scanning velocity: 0.01-10 cm/s; temperature of cooling: −20° C.-+30° C.

The optical element can also cooled by a thermal element(s) so as to prevent, or at least limit, heating of epidermis in the treatment area during irradiation. This cooling effect is also a function of the scanning velocity and is particularly critical where irradiation used is of a wavelength which preferentially targets melanin, as is for example the case for certain hair removal treatments. Since there is a high concentration of melanin at DE junction, it is desirable that V be slow enough so as to permit heat produced at the DE junction to be removed through the cooled waveguide or other cooled optically transparent element.

Cooling systems and phototreatment devices useful in conjunction with this invention are further described in U.S. Pat. No. 7,135,033, issued Nov. 14, 2006 entitled “Phototreatment device for use with coolants and topical substances” and U.S. Pat. No. 6,976,985 issued Dec. 20, 2005 entitled “Light energy delivery head” and U.S. application Ser. No. 10/154,756 filed May 23, 2002, entitled “Cooling System for a Photocosmetic Device,” the entirety of which are hereby incorporated by reference.

Further, as indicated earlier, the pressure applied to the skin by the head in general, and by the skin-contacting surface of the optical element in particular, has a number of advantages, including improving the optical transmission (i.e., reducing scattering) for radiation passing through the skin. The application of negative pressure to the treatment area stretches the skin resulting in an additional increase in skin transmission and thus the depth of electromagnetic wave penetration into the skin. Further, when the target is for example a hair follicle, the stretching of the skin turns the follicle to cause the radiation to impinge on a larger portion of the follicle and brings the follicle nearer to the skin surface.

Stretching of the skin also has additional benefits that are useful not only in photocosmetic treatments, but also when other stimuli are applied to the skin. These other stimuli can be used separately or in combination with light therapy. For example, stretching of the skin enhances application of current (AC or DC) through an electrical potential applied across the treatment area, application of ultra sound waves to the treatment area, and application of magnetic field through the treatment area. For example, an electrical current may be delivered at less potential and/or at higher current density due to the lowered contact resistance of stretched skin versus undisturbed skin. Although the above embodiments are directed to applicators that apply radiation to the skin, they can also be used to apply electrical current, acoustic energy, ultrasound, or other electrical currents or magnetic fields either alone or in conjunction with radiation. Descriptions of such applicators can be described in U.S. patent application Ser. No. 11/098,015, filed Apr. 1, 2005 and U.S. patent application Ser. No. 11/588,599 filed Oct. 27, 2006, which are incorporated herein by reference in their entirety.

### EXAMPLES

As described above, the methods and apparatus of the present invention can be used for a variety of photocosmetic methods including, but not limited to, acne treatment, skin rejuvenation, hair removal, cellulite treatment, fat reduction, wrinkle and scar reduction, collagen regeneration, tattoo removal, spider vein treatment, treatment of port-wine stains, and treatment of vascular lesions. The particular parameters will vary based on the desired treatment and characteristics of the patient, such as hair and skin color. The example below shows use of negative pressure with compressive contact for hair removal.

### Example

### Effect of Negative Pressure on Hair Removal

This Example shows the beneficial effects of using the methods of the invention for hair removal. The study was conducted as described below to evaluate the effect and safety of applying different levels of negative pressure to patients.

Excess or unwanted hair is a challenging problem, which may derive from inheritance, hormone imbalance, disease, or drugs. Currently, there are many methods of hair removal including shaving, wax epilation, chemical depilatories, electrolysis and hair removal using light (e.g. lasers or Intense Pulsed Light Sources (IPL)). The use of lasers or IPL devices is based on the principle of selective photothermolysis. Melanin in the hair shaft and/or follicles provides a chromophore absent in the dermis surrounding these follicles. Choosing a wavelength and pulse duration to selectively target melanin in the hair follicles will selectively destroy a large field of hair follicles while sparing the surrounding tissue. At deeply-penetrating wavelengths in the 600 nm-1100 nm region, melanin absorption is used for selective photothermolysis of hair follicles.

Long term, controlled hair counts indicate an average of 20 to 30% hair loss with each treatment, indicating the need for multiple treatments to obtain near complete hair removal. In this clinical study, a IPL device was used with suction feature on the handpiece to increase the efficacy of hair removal. The purpose of the vacuum system is to draw and stretch the skin into contact with the surface of the handpiece. This technique will effectively allow shorter distances between the desired target and the optical window, as well as decreased blood content in the treatment area, resulting in improved penetration of light to the target.

This study evaluated the response of hair removal with the IPL device with suction compared to standard IPL treatment. The study included 12 female subjects with skin of phototypes II-III, with an average aged 39.5 years (±8.8) with visible hair on axilla.

Mapping of Test Sites:

Overview pictures were taken of the test area. Immediately prior to IPL exposures, the test area was clipped to a length of about 2-3 mm and remaining clipped hairs removed by tape-stripping. Four test areas of 4 by 3 cm, were mapped on the thighs, back or axilla and photographed. The images provide hereby a permanent record of follicle density and individual hairs were counted from this set of images.

Light Exposures and Fluences:

Subjects and investigators wore protective eye wear. The test areas were shaved immediately before laser exposures and cleaned.

Three different treatment conditions were tested in each subject. The conditions of each test site are shown in table 3. The laser or IPL exposures were administered in adjacent exposure spots covering each of the test areas, at a repetition rate of 1 Hz.

TABLE 3
Treatment conditions.

| | Device | Fluence | Pulsewidth | Suction |
|---|---|---|---|---|
| 1. | Palomar | 75 J/cm² | 100 msec | Yes |
| | StarLux Rs | | | |
| 2. | Palomar | 75 J/cm² | 100 msec | No |
| | StarLux Rs | | | |
| 3. | Control | — | — | No |

Subjects returned 1, 3 and 6 months after the first set of IPL exposures. Responses were scored visually for changes in pigmentation, inflammation, hair regrowth and textural changes, using an arbitrary scale shown below (table 4). After evaluations were recorded, each skin site was clipped and photographed. These images allow for objective hair counting.

TABLE 4
Response Grading (subjective scale).

| | 0 | 1 | 2 | 3 |
|---|---|---|---|---|
| hair regrowth | none | sparse | moderate | full |
| hypopigmentation | absent | present | | |
| depigmentation | absent | present | | |
| hyperpigmentation | none | mild | moderate | severe |
| erythema | none | mild | moderate | severe |
| edema | none | mild | moderate | severe |
| purpura | none | mild | moderate | severe |
| textural change | none | mild | moderate | severe |
| (includes scarring) | | | | |
| ulceration | absent | present | | |

Skin Biopsies:

Subjects were asked to consent to have skin biopsies performed in the test sites. A total of 3 punch biopsies may be taken using standard aseptic technique.

Post-Exposure Skin Care:

After the light exposures, an antiseptic gel was applied. Subjects were instructed to gently clean the sites with warm water and mild soap, and to reapply the antiseptic gel twice a day for four days. Subjects were asked to avoid sun exposure or to use a SPF of 30 or higher for up to 3 months after treatment in order to reduce the risk of hyperpigmentation to the treatment sites.

Side Effects

The goal of this study is to selectively and permanently destroy hair follicles, with minimal or absent side effects. Alopecia is therefore the intended result in the test areas and in the treatment area. As with any form of laser or IPL treatment, there is a risk of infection, and/or scarring, which is minimized by proper wound care. Laser or IPL exposures could cause minimal changes in skin pigmentation. In addition, freckles may temporarily or permanently be removed in the laser exposed sites. Some subjects exposed to sunlight tend to heal with hyperpigmentation changes, and therefore avoidance of sun exposure at the treated skin sites is necessary during the study period. At the biopsy sites, a small scar will develop.

Follow-Up Schedule:

Hair regrowth and skin responses were recorded at 1 month, 3 months and 6 months after exposures in all test sites.

Materials:

The StarLux Pulsed Light System with the Rs handpiece is a broad spectrum, light-based device intended for the removal of unwanted hair and permanent hair reduction. The StarLux Rs handpiece delivers pulsed light with a wavelength of 600-1200 nm, a selectable pulse duration of 5-500 msec, and a fluence range up to 85 J/cm². The light pulses are generated at a frequency of 0.5-1 Hz and delivered through a spot size of 12×28 mm. The handpiece tip is water cooled for direct contact cooling of the skin. A vacuum system attachment was used for the purpose of drawing the skin closer to the optical window. The pressure in the vacuum chamber is approximately 0.3-0.9 bar.

Number of Subjects:

The goal of the study is to compare the efficacy of hair removal by single treatment by conventional IPL devices compared to IPL with suction, to shaved-only and to suction-only control site. An important outcome variable is the percentage of hair loss at the 6 month follow-up visit. Our previous study found a statistically significant 30% mean hair loss with a standard deviation of about 5% after 1 ruby laser treatment in 12 subjects. In order to detect a difference of 10% in mean hair loss between the different conditions with 5% standard deviation, a minimum of 8 patients is required. This calculation is based on a=0.05, b=0.05, and a two-sided comparison. A 50% loss due to drop out or loss to follow-up requires up to 12 subjects enrolled at start.

Data Analysis:

Photographs of regrowing hairs at 1, 3 and 6 months against the baseline hair follicle images were used to establish the fraction of regrowing hairs as a function of exposure conditions. For the comparison of the percentage of hair regrowth for each exposure condition to the baseline, a simple t-test for independent variables were used.

Results

All patients showed immediate perifollicular erythema/edema. The test sites with Rs suction showed purpura around irradiated skin. No hypopigmentation, hyperpigmentation, or textural change/scaring was seen in any of the test subjects.

The results experiment at 1, 3 and 6 months following light exposure are shown below in Table 5 as percentage of hair growth:

TABLE 5
Results of treatment at 1, 3 and 6 months following light exposure.

| | | | | Paired t-test |
|---|---|---|---|---|
| Time after | | StarLux Rs (no | StarLux Rs (with | (Rs suction vs. |
| treatment | Control | suction) | suction) | Rs no suction) |
| 1 month | 101.2% | 30.6% | 42.8% | 0.026 |
| 3 month | 101.9% | 59.8% | 62.6% | 0.593 |
| 6 month | 98.1% | 59.7% | 55.1% | 0.328 |

The results indicate that the addition of suction reduced hair regrowth at six months after treatment. By varying the parameters based on the patient's skin and hair type improved effects can be achieved.

### EQUIVALENTS

One skilled in the art will appreciate further features and advantages of the invention based on the above-described embodiments. Accordingly, the invention is not to be limited by what has been particularly shown and described, nor by the examples set forth below, except as indicated by the appended claims. While only certain embodiments have been described, it will be understood by those skilled in the art that various changes in form and details may be made therein without departing from the spirit and scope as defined by the appended claims. Those skilled in the art will recognize, or be able to ascertain using no more than routine experimentation, many equivalents to the specific embodiments described specifically herein. Such equivalents are intended to be encompassed in the scope of the appended claims.

The patent, scientific and medical publications referred to herein establish knowledge that was available to those of ordinary skill in the art at the time of the invention. The entire disclosures of the issued U.S. patents, published and pending patent applications, and other references cited herein are hereby incorporated by reference.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art.

As used herein, EMR includes the range of wavelengths approximately between 200 nm and 10 mm. Optical radiation, i.e., EMR in the spectrum having wavelengths in the range between approximately 200 nm and 100 μm, is preferably employed in some of the embodiments described above, but, also as discussed above, many other wavelengths of energy can be used alone or in combination. Also as discussed, wavelengths in the higher ranges of approximately 2500-3100 nm may be preferable for creating micro-holes using ablative techniques. Additionally, the term optical (when used in a term other than term “optical radiation”) applies to the entire EMR spectrum. For example, as used herein, the term “optical path” is a path suitable for EMR radiation other than “optical radiation.”

It should be noted, however, that other energy may be used to for treatment islets in similar fashion. For example, sources such as ultrasound, photo-acoustic and other sources of energy may also be used in some embodiments. Thus, although the embodiments described herein generally are described with regard to the use of EMR, other forms of energy are within the scope of the invention and the claims.

1. A method for treating a volume of tissue, comprising:
applying a negative pressure to at least a portion of the volume of tissue;
mechanically restraining a second portion of the volume of tissue; and
irradiating the second portion of the volume with energy.

2. The method of claim 1, wherein the second portion is mechanically restrained by the surface of an energy-transmissive element through which the second portion is irradiated with the energy.

3. The method of claim 1, wherein the radiant energy is at least one form of energy from the group of electromagnetic radiation, acoustic energy, electric current, and heat.

4. The method of claim 1, wherein the first and second portions do not overlap.

5. The method of claim 1, wherein at least part of the first and second portions of the volume of tissue overlap.

6. The method of claim 1, further comprising cooling the volume of tissue.

7. The method of claim 6, wherein the cooling step comprises cooling the end of the optical element that is in contact with the volume of tissue.

8. The method of claim 1, wherein the step of applying negative pressure further comprises stretching the volume of tissue.

9. The method of claim 8, wherein the volume of tissue is stretched for an amount of time sufficient to reduce the amount of blood in the volume of tissue.

10. The method of claim 1, wherein the step of applying negative pressure further comprises applying negative pressure in a range of about 6.7×10³ Pa to about 1×10⁵ Pa.

11. The method of claim 1, wherein the negative pressure is in a range of about 23×10³ Pa to about 41×10³ Pa.

12. The method of claim 1, wherein the negative pressure is applied for a duration of about 1 milliseconds to 2 seconds.

13. The method of claim 1, wherein the energy is electromagnetic radiation and the method further comprises selecting one or more wavelengths of the radiation so as to perform any of acne treatment, skin rejuvenation, hair removal, cellulite treatment, fat reduction, wrinkle and scar reduction, collagen regeneration, tattoo removal, and treatment of pigmented and vascular lesions.

14. The method of claim 1, wherein the energy is electromagnetic radiation that includes at least one wavelength in a range of about 300 nm to about 11,000 nm.

15. The method of claim 1, wherein the energy is electromagnetic radiation that includes at least one wavelength in a range of about 300 nm to about 3,000 nm.

16. The method of claim 1, wherein the energy is electromagnetic radiation delivered to the volume of tissue at a power density in a range of about 1 mW/cm² to about 1000 W/cm² to the volume of tissue.

17. The method of claim 1, wherein the energy is electromagnetic radiation delivered to the volume of tissue at a power density in a range of about 100 mW/cm² to about 10 W/cm² to the volume of tissue.

18. The method of claim 1, wherein the energy is electromagnetic radiation delivered to the volume of tissue at a fluence in a range of about 1 J/cm² to about 1000 J/cm².

19. The method of claim 1, wherein the energy is electromagnetic radiation delivered to the volume of tissue at a fluence in a range of about 10 J/cm² to about 500 J/cm².

20. The method of claim 1, wherein the optical element comprises a radiation-transmissive block.

21. The method of claim 1, further comprising releasing the negative pressure after application of the radiation to the volume of tissue.

22. The method of claim 1, further comprising moving an energy-transmissive element to another volume of tissue by sliding the element from a surface of the first volume to a surface of the second volume.

23. The method of claim 22, further comprising irradiating tissue with energy during the transition from the first volume to the second volume.

24. The method of claim 1, further comprising compressing a third portion of the volume of tissue.

25. The method of claim 22, wherein at least some of the third portion of the volume of tissue is contiguous with at least some of the second portion of the volume of tissue that is mechanically restrained.

26. The method of claim 1, further comprising moving the element to a second volume of tissue.

27. The method of claim 26, further comprising:
applying a negative pressure to at least a portion of the second volume of tissue;
mechanically restraining a second portion of the second volume of tissue; and
irradiating the second portion of the second volume of tissue with electromagnetic radiation.

28. The method of claim 27, wherein the step of moving is accomplished by stamping each volume of tissue being treated.

29. The method of claim 1, further comprising:
mechanically restraining a third portion of the volume of tissue; and
irradiating the third portion of the volume of tissue with electromagnetic radiation;
wherein the second and third portions of the volume of tissue are not contiguous.

30. The method of claim 1, wherein the step of irradiating further comprises simultaneously irradiating a plurality of portions of the volume of tissue, wherein each portion of the plurality is spaced a distance from the other portions of the plurality.

31. The method of claim 1, further comprising monitoring the negative pressure to ensure it remains below a pre-defined threshold.

32. The method of claim 31, wherein the step of monitoring the negative pressure further comprises adjusting the pressure based on a selected treatment for the volume of tissue.

33. The method of claim 1, further comprising applying a positive pressure to at least a third portion of the volume of tissue.

34. The method of claim 33, wherein the third portion does not overlap the first or second portions.

35. The method of claim 33, wherein the third portion overlaps at least one of the first and second portions.

36. The method of claim 1, further comprising applying pressure, wherein the pressure is alternated between positive and negative pressure.

37. The method of claim 1, wherein negative pressure is applied such that the volume of tissue is stretched in a first direction.

38. The method of claim 37, further comprising applying negative pressure such that the volume of tissue is stretched in a second direction.

39. The method of claim 38, wherein the volume of tissue is alternatingly stretched in the first direction and then the second direction.

40. A photocosmetic method, comprising
placing an optically transmissive surface in proximity of a skin region,
applying a negative pressure to the skin region in order to draw a portion thereof into contact with the optical surface so as to redistribute blood volume between the skin portion in contact with the optical surface and the remainder of the skin region, and
applying radiation through the surface to the skin portion.

41. The method of claim 40, wherein the redistribution of the blood volume is characterized by a decrease in volumetric blood concentration at the skin portion in contact with the surface.

42. The method of claim 40, wherein the redistribution of the blood volume is characterized by an increase in volumetric blood concentration in the remainder of the skin region.

43. The method of claim 40, further comprising selecting the negative pressure such that the skin portion in contact with the surface substantially covers the surface.

44. The method of claim 43, further comprising selecting the negative pressure such that the skin portion substantially conforms to a topographical profile of the surface.

45. The method of claim 40, further comprising cooling the surface.

46. The method of claim 40, wherein the negative pressure causes stretching of the skin portion.

47. A method of dermatological treatment, comprising
applying negative pressure to a plurality of surface skin segments within a skin region so as to redistribute blood within the region,
applying radiation to the skin region, wherein the blood redistribution causes a non-uniform absorption of the radiation across the skin region.

48. The method of claim 41, wherein the non-uniform absorption comprises increased absorption at one or more skin targets located below surface skin segments.

49. The method of claim 47, wherein the method further comprises monitoring the negative pressure applied to the skin segments.

50. The method of claim 47, wherein the method further comprises adjusting the negative pressure and radiation based on a desired radiation pattern corresponding to a desired treatment of the skin region.

51. A dermatological treatment method, comprising
placing an optical surface in proximity of a skin region containing a skin target, the optical surface being at least partially surrounded by a negative pressure chamber,
applying a negative pressure to the skin region so as to draw the skin target into contact with the optical surface causing redistribution of blood volume between the skin target and the remainder of the skin region, and
applying radiation through the surface to the skin target.

52. An dermatological device, comprising
an optical element adapted for contact at one end thereof with a skin target,
a negative pressure chamber at least partially surrounding the end of the optical element, wherein the negative pressure chamber is adapted to apply a negative pressure to one or more locations of a skin region so as to draw the skin target into compressive contact with the end of the optical element and to cause a depletion of blood volume within the skin target.

53. The device of claim 52, wherein negative pressure applied to the skin region is in a range of about 6.7×10³ Pa to about 1×10⁵ kPa.

54. The device of claim 52, wherein the negative pressure chamber is adapted to apply a negative pressure along an axial direction to the skin.

55. The device of claim 54, wherein the axial negative pressure causes a transverse stretching of the skin target.

56. The device of claim 54, wherein the device further includes means for controlling the negative pressure.

57. The device of claim 54, wherein the negative pressure chamber comprises a plunger.

58. The device of claim 54, wherein the negative pressure chamber is coupled to a negative pressure source.

59. The device of claim 58, wherein the device further includes a pressure sensor and a feedback loop between the pressure sensor and the source of negative pressure.

60. The device of claim 54, wherein the device further includes a radiation source capable of irradiating through the optical element.

61. The device of claim 58, wherein the feedback loop is adapted to activate a radiation source in response to a detected pressure.

62. The device of claim 58, wherein the device comprises a pressure release valve.

63. The device of claim 62, wherein the device further comprises a pressure controller.

64. A dermatological device, comprising
an element configured to transmit electromagnetic radiation and further configured to be in contact with a skin target for applying electromagnetic radiation thereto,
a channel extending from a proximal end adapted for coupling to a pressure source to a distal end defining a pressure chamber and configured to apply a pressure to a skin region containing at least one skin portion offset from the skin target,
wherein at least a portion of the element is located within the pressure chamber.

65. The dermatological device of claim 58, wherein the pressure source is further capable of applying a positive pressure.

66. The dermatological device of claim 64, wherein the pressure source is further capable of applying a negative pressure to the skin portion so as to cause stretching of the skin target thereby depleting blood volume therein.

67. The dermatological device of claim 64, wherein the distal end of the channel is axially offset from the distal end of the element.

68. The dermatological device of claim 64, wherein the distal end of the channel is substantially flush with the distal end of the element.

69. The dermatological device of claim 64, wherein the distal end of the channel is surrounded by an inflatable cuff capable of increasing tension of the skin target.

70. An adapter for use with a photocosmetic device, comprising
a pressure applicator assembly adapted to removeably and replaceably couple to a distal end of a waveguide of the device, the coupling comprising a seal between the assembly and the distal end, the assembly comprising,
a negative pressure chamber at a distal end thereof adapted for coupling to a skin portion to apply a negative pressure thereto, and
at least one channel extending from a proximal end adapted for coupling a source of negative pressure to the distal end having an opening to the chamber.

71. The adapter of claim 70, wherein the pressure applicator further comprises a pressure sensor.

72. The adapter of claim 71, wherein the pressure sensor is coupled to a pressure control valve.

73. A method of treating the skin, comprising
placing a surface of a radiation-transmissive element in contact with a skin target,
applying a negative pressure to a periphery of the skin target so as to cause stretching thereof, and
applying radiation through the surface to the skin in contact therewith.

74. A dermatological device, comprising
an optical waveguide adapted for contact at one end thereof with a skin target,
a skin pressure applicator coupled to the end of the waveguide, the applicator comprising
a first channel adapted for coupling at a proximal end to a source of positive pressure and for applying at a distal end a positive pressure to a first skin region,
a second channel adapted for coupling at a proximal end to a source of negative pressure and for applying at a distal end a negative pressure to a second skin region.

75. The dermatological device of claim 74, wherein the skin regions are offset relative to the skin target.

76. The dermatological device of claim 74, wherein the skin regions partially surround the skin target.

77. The dermatological device of claim 74, wherein the pressures are selected to hold the skin target under tension in contact with the end of the optical waveguide.

78. A dermatological device, comprising
a housing providing an optical path extending from a proximal end thereof to a distal end for applying radiation to a skin region,
a skin pressure applicator coupled to the distal end of the housing for applying pressure to the skin region, the pressure applicator comprising
a pressure mask having a plurality of openings to allow application of pressure to a plurality of locations of the skin region so as to non-uniformly redistribute blood volume.

79. The dermatological device of claim 78, wherein the pressure applicator further comprises a pressure chamber.

80. The dermatological device of claim 79, wherein the pressure chamber is coupled to a negative pressure source.

81. The device of claim 80, wherein the pressure causes stretching of the skin regions.

82. The device of claim 80, wherein the pressure mask is comprised of an optically transmissive material.

83. The dermatological device of claim 79, wherein the pressure chamber is coupled to a positive pressure source.

84. A method for treating a volume of tissue, comprising:
applying a negative pressure to at least a portion of the volume of tissue;
compressing a second portion of the volume of tissue; and
irradiating the second portion of the volume with electromagnetic radiation.
